(19) United States
(12) Patent Application Publication
(10) Pub. No.: US 2026/0294954 A1
(43) Pub. Date: Oct. 1, 2026

(54) APTAMER-BASED SUSTAINED RELEASE OF THERAPEUTIC AGENTS

(71) Applicant: The Children's Medical Center Corporation, Boston, MA (US)

(72) Inventors: Christopher B. Weldon, Brookline, MA (US); Daniel S. Kohane, Newton, MA (US); Dali Wang, Malden, MA (US); Xiangang Huang, Cambridge, MA (US)

(73) Assignee: The Children's Medical Center Corporation, Boston, MA (US)

(21) Appl. No.: 19/477,881

(22) PCT Filed: Apr. 23, 2024
(86) PCT No.: PCT/US2024/025776

§ 371 Date: Oct. 23, 2025

Related U.S. Application Data
(60) Provisional application No. 63/461,525, filed on Apr. 24, 2023.

(51) Int. Cl. A61K 31/711; A61K 31/137; A61K 31/4045; A61K 31/519; A61K 31/52; A61K 31/7036; A61K 38/28; A61P 25/00

(52) U.S. Cl. CPC A61K 31/711; A61K 31/137; A61K 31/4045; A61K 31/519; A61K 31/52; A61K 31/7036; A61K 38/28; A61P 25/00

(57) ABSTRACT

The present disclosure provides compositions comprising an aptamer and a molecular payload, wherein the aptamer is a sustained release carrier for the molecular payload. The molecular payload may be a therapeutic agent. The compositions thus provide a means of controlled and prolonged delivery of therapeutic agents. The present disclosure further provides kits comprising the compositions, methods of treating or preventing a disease or disorder, and methods of preparing the compositions.

### RELATED APPLICATIONS

This application claims priority under 35 U.S.C. § 119(e) to U.S. Provisional Application, U.S. Ser. No. 63/461,525, filed Apr. 24, 2023, which is incorporated herein by reference in its entirety.

### FEDERALLY SPONSORED RESEARCH

This invention was made with government support under Grant Number GM131728, awarded by the National Institutes of Health. The Government has certain rights in the invention.

### BACKGROUND

Aptamers can bind to small molecules with high affinity and specificity. Their affinity and selectivity to target molecules rivals those of antibodies, and they do not have demonstrable biological toxicity. They also have been shown to have limited or no immunogenicity. Furthermore, they do not have sequence homology to coding or non-coding genetic sequences of any organism. These attributes have enabled aptamers to be utilized in a wide range of applications, including diagnostics, biosensor technologies, affinity isolation, biomarker discovery, and targeted therapeutics. Aptamers have been used as the targeting ligands for a variety of systemically delivered drug delivery systems. However, this approach is limited to drugs that can interact with aptamers by nonspecific interactions such as charge and hydrophobicity, and/or wherein the aptamer functions as a targeting moiety to locate the drug to a specific biological target.

### SUMMARY

The present disclosure stems from the recognition that aptamers can function as sustained release carriers for drug delivery and other applications by taking advantage of the binding of an aptamer to a molecular payload. The binding of aptamer to molecular payload prolongs duration of release of the payload (and therefore duration of effect), while reducing systemic toxicity associated with the molecular payload. Thus, the disclosed compositions and methods provide a new platform for the controlled delivery of therapeutic agents in a variety of settings and for the treatment of a range of diseases and/or conditions.

In one aspect, provided are compositions comprising an aptamer and a molecular payload, wherein the molecular payload is bound to the aptamer, and wherein the aptamer is a sustained release carrier for the molecular payload.

In another aspect, provided are methods of treating a disease or condition, the method comprising administering an effective amount of the composition.

In another aspect, provided are methods of preparing the composition, the method comprising providing a molecular payload; identifying or preparing an aptamer that binds (e.g., specifically binds) to the molecular payload; and combining the molecular payload and aptamer in a composition.

In another aspect, provided are kits comprising the composition and instructions for using the composition.

The details of certain embodiments of the invention are set forth in the Detailed Description of Certain Embodiments, as described below. Other features, objects, and advantages of the invention will be apparent from the Definitions, Drawings, Examples, and Claims.

### Definitions

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease or disorder.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease or disorder described herein. In some embodiments, treatment may be administered after one or more signs or symptoms of the disease or disorder have developed or have been observed. In other embodiments, treatment may be administered in the absence of signs or symptoms of the disease. For example, treatment may be administered to a susceptible subject prior to the onset of symptoms (e.g., in light of a history of symptoms). Treatment may also be continued after symptoms have resolved, for example, to delay or prevent recurrence.

The term “prevent,” “preventing,” or “prevention” refers to a prophylactic treatment of a subject who is not and was not with a disease or disorder but is at risk of developing the disease or disorder or who was with a disease or disorder, is not with the disease or disorder, but is at risk of regression of the disease or disorder. In certain embodiments, the subject is at a higher risk of developing the disease or disorder or at a higher risk of regression of the disease or disorder than an average healthy member of a population of subjects.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient, e.g. for inhibiting a biological target (e.g., at least 5%, at least 10%, at least 20%, at least 30%, at least 40%, at least 50%, at least 60%, at least 70%, at least 80%, at least 90%, at least 95%, or at least 99% inhibition of the target). In certain embodiments, a therapeutically effective amount is an amount sufficient for treating a disease or condition.

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for inhibiting expression of a target nucleic acid. In certain embodiments, a prophylactically effective amount is an amount sufficient for treating a disease or disorder.

The term “molecular payload” refers to a molecule or species that functions to modulate and/or induce a biological outcome.

The term “nucleic acid” refers to biopolymers, macromolecules, essential to all known forms of life. Nucleic acids comprise nucleotides, which are its monomers made of three components: a 5-carbon sugar, a phosphate group and a nitrogenous base. The two main classes of nucleic acids are deoxyribonucleic acid (DNA) and ribonucleic acid (RNA).

The terms “nucleoside” and “nucleotide” refer to moieties which contain not only the known purine and pyrimidine bases, but also other heterocyclic bases which have been modified. In certain embodiments, the nucleobase is replaced with a moiety that is not a nucleobase. Modified nucleosides or nucleotides can also include modifications on the sugar moiety, e.g., wherein one or more of the hydroxyl groups are replaced with hydrogen, halogen (e.g., fluoro), aliphatic groups, or are functionalized as ethers or amines. The term “nucleotidic unit” is intended to encompass nucleosides and nucleotides as well as modified forms of both.

The term “polynucleotide” as used herein includes polymers of deoxyribose nucleic acids (DNA) or ribose nucleic acids (RNA) having the standard nucleotide bases A, G, T, and C for DNA or in the case of RNA A, G, C, and U, a subset of the standard nucleotide bases that uses less than four of the standard nucleotide bases, and unnatural bases such as 7-(2-thienyl)imidazo[4,5-b]pyridine (Ds), pyrrole-2-carbaldehyde (Pa), 2-amino-8-(2-thienyl)purine (s), f 2-amino-6-(N,N-dimethylamino)purine (x), pyridine-2-one (y), 3-nitropyrrole, 5-nitroindole, and 4-[3-(6-aminohexanamido)-1-propynyl]-2-nitropyrrole (Px) or other unnatural bases currently known or subsequently discovered. Examples provided in this disclosure may reference DNA or RNA, or any of the nucleotide bases A, G, T, C, and U. However, this is to be understood as merely illustrative and does not exclude implementations using RNA, DNA-RNA hybrids, and/or unnatural bases. In certain embodiments, a polynucleotide may include a chemically modified nucleotide.

The term “oligonucleotide” refers to an oligomeric nucleic acid compound of up to 200 nucleotides in length. Examples of oligonucleotides include, but are not limited to, DNA, RNA, RNAi oligonucleotides (e.g., siRNAs, shRNAs), microRNAs, gapmers, mixmers, phosphorodiamidite morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleotides (e.g. 2′-O-methyl sugar modifications, purine or pyrimidine modifications). In some embodiments, an oligonucleotide may comprise one or more modified internucleotide linkage. In some embodiments, an oligonucleotide may comprise one or more phosphorothioate linkages, which may be in the Rp or Sp stereochemical conformation.

The term “sequence” or “nucleotide sequence” mean a succession or order of nucleobases or nucleotides, described with a succession of letters using the standard nucleotide nomenclature.

As used herein, and unless otherwise indicated, the term “complementary,” when used to describe a first nucleotide sequence in relation to a second nucleotide sequence, means the ability of an oligonucleotide or polynucleotide including the first nucleotide sequence to hybridize (form base pair hydrogen bonds under mammalian physiological conditions (or comparable conditions in vitro)) and form a duplex or double helical structure under certain conditions with an oligonucleotide or polynucleotide including the second nucleotide sequence. Complementary sequences include Watson-Crick base pairs or non-Watson-Crick base pairs and include natural or modified nucleotides or nucleotide mimics, at least to the extent that the above requirements with respect to the ability to hybridize are fulfilled.

As used herein, “perfectly complementary” or “fully complementary” means that all (100%) of the bases in a contiguous sequence of a first polynucleotide will hybridize with the same number of bases in a contiguous sequence of a second polynucleotide. The contiguous sequence may comprise all or a part of a first or second nucleotide sequence.

As used herein, “partially complementary” means that in a hybridized pair of nucleobase sequences, at least 70%, but not all, of the bases in a contiguous sequence of a first polynucleotide will hybridize with the same number of bases in a contiguous sequence of a second polynucleotide.

As used herein, “substantially complementary” means that in a hybridized pair of nucleobase sequences, at least 85%, but not all, of the bases in a contiguous sequence of a first polynucleotide will hybridize with the same number of bases in a contiguous sequence of a second polynucleotide.

The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities, such as cells and tissues. Biologics may be isolated from a variety of natural sources (e.g., human, animal, microorganism) and may be produced by biotechnological methods and other technologies.

The term “small molecule” or “small molecule therapeutic” refers to molecules, whether naturally occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

The terms “therapeutic compound,” “therapeutic agent,” or “therapeutic moiety” refer to any substance having therapeutic properties that produce a desired, usually beneficial, effect. For example, therapeutic compounds, agents, and moieties may treat and/or ameliorate a disease or disorder. Therapeutic compounds, agents, and moieties, as disclosed herein, may be biologics or small molecule therapeutics, or combinations thereof.

The term “sustained release carrier” refers to an entity capable of associating with, interacting with, chaperoning, and/or facilitating the delivery of a molecular payload (e.g., therapeutic agent) to a desired location in the body of a subject. The sustained release carrier may facilitate local sustained release of a molecular payload. The sustained release carrier may facilitate systemic sustained release of a molecular payload. A sustained release carrier may release the molecular payload slowly over time to permit less frequent administration of the molecular payload. A sustained release carrier may provide longer duration of a therapeutic effect through slow absorption into the bloodstream and/or tissue.

The term “depot” refers to a composition or formulation comprising a molecular payload which releases the molecular payload slowly over time, typically placed at a single location in the body. Depots are typically administered intramuscularly or subcutaneously and release a molecular payload to a local or systemic site.

The term “sustained release” (also referred to as extended release or controlled release) refers to a continuous or continual release of a molecular payload introduced into the body of a subject over a period of time and at a therapeutic level sufficient to achieve a desired therapeutic effect throughout the period of time. The rate at which the molecular payload is released is slower than the rate of release of the molecular payload when it is administered alone or in a non-sustained release formulation. Sustained release formulations may, by way of example, be created as films, slabs, pellets, microparticles, microspheres, microcapsules, spheroids, shaped derivatives and paste. The formulations may be in a form that is suitable for suspension in isotonic saline, physiological buffer or other solution acceptable for injection into a patient. Further, the formulations may be used in conjunction with any implantable, insertable or injectable system that a person of ordinary skill would appreciate as useful in connection with embodiments herein including but not limited to parenteral formulations, microspheres, microcapsules, gels, pastes, implantable rods, pellets, plates or fibers, etc.

### BRIEF DESCRIPTION OF THE DRAWINGS

FIGS. 1A-1E show aptamers for drug delivery. FIG. 1A is a schematic of aptamer creation, binding of a drug, (site 1 sodium channel blocker (S1SCB) used as the example), then release in vivo where the drug has an effect. FIG. 1B shows chemical structure units of S1SCB-binding aptamers. PO: phosphodiester; PS: phosphorothioate. FIG. 1C shows the chemical structures of tetrodotoxin (TTX), saxitoxin (STX) and bupivacaine hydrochloride (HCl). FIG. 1D shows TTX binding affinity analysis by microscale thermophoresis of TTX-binding aptamers (PO and PS), and an aptamer with a scrambled sequence (Scr-PS). Y-axis shows the fraction of TTX bound to the aptamer. n=3 independent experiments. FIG. 1E shows cumulative TTX release from aptamer/TTX complexes and controls at 12 h. The TTX concentration for each group was 42 μM. Data are shown as the mean±s.d., n=4 independent experiments. Statistical analysis was performed using one-way ANOVA with Tukey's multiple comparison testing. NS, not statistically significant.

FIGS. 2A-2H show peripheral nerve blocks with PS/TTX complexes. FIG. 2A shows a peripheral nerve blockade with 42 μM TTX, free or complexed with aptamers (PO, PS, Scr-PS) (n=6 biologically independent animals for TTX and PS/TTX; n=4 biologically independent animals for PO/TTX and Scr-PS/TTX). FIG. 2B shows a peripheral nerve blockade with 22 μM STX alone or combined with the TTX-specific PS aptamer (n=4 biologically independent animals for STX and PS/STX). FIG. 2C shows a peripheral nerve blockade with bupivacaine alone or combined with the TTX-specific PS aptamer (n=6 biologically independent animals for bupivacaine and PS/bupivacaine). The bupivacaine concentration in each formulation was 15.4 mM. FIG. 2D shows a peripheral nerve blockade with varying molar ratios of PS/TTX complexes, using 42 μM TTX. Data are means±s.d. (n=6 biologically independent animals for TTX and PS/TTX 20:1; n=4 biologically independent animals for PS/TTX 1:1, 2:1, 5:1, 10:1, and 40:1). FIG. 2E shows a sciatic nerve blockade with free TTX and PS/TTX (2:1) in the injected (left panel) and contralateral (right panel) hind paws. The dagger indicates 100% mortality. Data are means±s.d., n=6 biologically independent animals for TTX 42 μM and TTX 52 μM; n=4 biologically independent animals for TTX 31 μM, TTX 63 μM, and PS/TTX groups. FIG. 2F shows the frequency of nerve block in the contralateral (uninjected) legs (n=6 biologically independent animals for TTX 42 μM and TTX 52 μM; n=4 biologically independent animals for TTX 31 μM and PS/TTX groups). FIG. 2G shows animal mortality after treatment with TTX and PS/TTX (2:1) (n=6 biologically independent animals for TTX 42 μM and TTX 52 μM; n=4 biologically independent animals for TTX 31 μM, TTX 63 μM, and PS/TTX groups). FIG. 2H shows the effect of 55 μM epinephrine on the duration of sensory nerve blockade from PS/TTX (2:1, 73 μM, 84 μM and 104 μM TTX). Data are means±s.d., n=4 rats per group. Statistical comparisons were performed using Student t-test (two-sided).

FIGS. 3A-3C shows the tissue distribution of a PS aptamer. FIG. 3A shows representative time courses for the retention of Cy5.5 labelled aptamers (PO or PS) or free Cy5.5 at the site of injection as monitored by an IVIS. Color represents fluorescence from Cy5.5. FIG. 3B shows a quantification of the fluorescence intensity over time (as a percentage of intensity at time=0, immediately after sciatic nerve injection), derived from data in FIG. 3A. Data are means±s.d., n=4 rats per group. Statistical comparisons were performed using Student t-test (two-sided). FIG. 3C shows representative confocal images of the rat sciatic nerve and surrounding tissue cryosections 4 h after sciatic nerve injection of Cy5.5 or Cy5.5-labelled aptamers (PO and PS). Each experiment was repeated three times independently with similar results.

FIG. 4 shows tissue reaction to free TTX, PS aptamer, and PS/TTX complexes. H&E: Representative hematoxylin-eosin stained sections of muscles and adjacent loose connective tissue 4 and 14 days after sciatic nerve injection of TTX (52 μM), PS aptamer (146 μM), or PS/TTX (2:1, 73 μM TTX) in 0.3 mL of PBS. Toluidine Blue: Representative toluidine blue stained sections of nerve 4 days and 14 days after sciatic nerve injection of the above formulations. n=4 in each group.

FIGS. 5A-5B show the peripheral nerve blockade effect of STX-binding PS aptamer (PSAP_{STX})/STX. FIG. 5A shows the duration of sensory nerve blocks in the injected legs and uninjected (contralateral) extremities after sciatic nerve injection (n=4 biologically independent animals). Data are means±s.d. Statistical comparisons were performed using Student t-test (two-sided). FIG. 5B shows tissue reaction to STX (33 μM), PSAP_{STX} (90 μM), and PSAP_{STX}/STX (2:1, 45 μM STX) complexes 4 and 14 days after administration.

FIGS. 6A-6B show a comparison of aptamer size. FIG. 6A shows the size of aptamer/TTX conjugates at different molar ratios of aptamers to TTX, as measured by dynamic light scattering (DLS). The TTX concentration was fixed (42 μM). Data are means±s.d., n=3 independent experiments. Statistical comparisons were performed using Student t-test (two-sided). FIG. 6B shows representative number-average size distribution of aptamer/TTX.

FIGS. 7A-7B show TTX release kinetics from aptamer/TTX conjugates and controls over 24 h. FIG. 7A shows free TTX versus PO/TTX versus PS/TTX. FIG. 7B shows free TTX versus Scr-PS/TTX. The TTX concentration for each group was 42 μM. Data are shown as the mean±s.d., n=4 independent experiments. Statistical comparisons were performed using Student t-test (two-sided). NS, not statistically significant.

FIGS. 8A-8B show results of MTS cytotoxicity assays for (FIG. 8A) myotoxicity (in C2C12 cells) and (FIG. 8B) neurotoxicity (in PC12 cells) after 24 h exposure to the following groups: free TTX, PO aptamer, PS aptamer, PO/TTX (2:1), or PS/TTX (2:1). The TTX concentration was 73 μM and the aptamer concentration was 146 μM. Data are means±s.d., n=4 biological replicates per group.

FIGS. 9A-9D show representative time courses of thermal latency after sciatic nerve injections of aptamer formulations. Rats were injected with 0.3 mL of (FIG. 9A) free TTX, (FIG. 9B) PO/TTX (20:1), (FIG. 9C) PS/TTX (20:1), or (FIG. 9D) scrambled PS/TTX (20:1) in PBS, all with 42 μM TTX. Data are means±s.d., n=4 biologically independent animals per group.

FIGS. 10A-10B show representative time courses of thermal latency after sciatic nerve injections of (FIG. 10A) PO aptamer or (FIG. 10B) PS aptamer. All injections were in 0.3 mL of PBS at an aptamer concentration of 836 μM. Data are means±s.d., n=4 biologically independent animals per group.

FIG. 11 shows viscosities of the PS/TTX (20:1) and Scr-PS/TTX (20:1). The concentration of TTX was 42 μM. There was no statistically significant difference between these two groups (n=3, p>0.05). Data are means±s.d., n=3 independent experiments. Statistical comparisons were performed using Student t-test (two-sided).

FIG. 12 shows viscosities of the PS aptamer alone (84 μM) and differing molar ratios of PS aptamer to TTX. The concentration of TTX was 42 μM. There was no difference in the measured viscosity of the various ratios (n=3, all p>0.05 for all comparisons). Data are means±s.d., n=3 independent experiments. Statistical comparisons were performed using Student t-test (two-sided).

FIG. 13 shows a representative comparison of the duration of sensory and motor blocks. The rats were injected with free TTX or PS/TTX (2:1) in 0.3 mL of PBS. The TTX concentration for each group has been indicated in the figure. P values are for the comparison of the duration of sensory block of formulations to that of motor block. Data are means±s.d.; n=4 biologically independent animals per group. Statistical comparisons were performed using Student t-test (two-sided).

FIG. 14 shows representative time courses of thermal latency after sciatic nerve injections of PS/TTX (2:1, 73 μM TTX) or in combination with 55 μM epinephrine. Data are means±s.d.; n=4 biologically independent animals per group.

FIG. 15 shows representative photographs of dissected sciatic nerve (white arrow) and surrounding tissues of rats 4 hours after sciatic nerve injection. The blue color is from Cy5.5.

FIG. 16 shows confocal images of the rat sciatic nerve and surrounding tissue cryosections 4 h after sciatic nerve injection of Cy5.5-labelled PS aptamer. Each experiment was repeated three times independently with similar results.

FIG. 17 shows cytotoxicity of STX, STX-binding PS aptamer (PSAP_{STX}), and PSAP_{STX} conjugates in C1C12 and PC12 cells. The STX concentration was 45 μM and the PSAP_{STX} concentration was 90 μM. The molar ratio of PSAP_{STX}:STX=2:1. Data are means±s.d.; n=4 biological replicates per group.

FIG. 18A-C shows aptamers (Apt) for sustained release of different drugs. Different drug-binding aptamers, including serotonin (Ser)-binding Apt (AptSer), kanamycin (Kan)-binding Apt (AptKan) and insulin (Ins)-binding Apt (AptIns), were utilized in this study. FIG. 18A: In vitro drug release profile of Ser from Apt-Ser complexes (top) and cumulative release of AptSer from its corresponding Apt-Ins complex (bottom). FIG. 18B: In vitro drug release profile of Kan from Apt-Kan complexes (top) and cumulative release of AptKan from its corresponding Apt-Ins complex (bottom). FIG. 18C: In vitro drug release profile of Ins from Apt-Ins complexes (top) and cumulative release of AptIn from its corresponding Apt-Ins complex (bottom). n=4 independent experiments. Data are presented as mean±s.d. Statistical significance was evaluated by one-way ANOVA with a Tukey post hoc test in a, b, c, d, e, f. *P<0.05, **P<0.01, ****P<0.0001.

### DETAILED DESCRIPTION OF CERTAIN EMBODIMENTS

Herein, it is demonstrated that non-covalent complexation of molecular payloads (e.g., therapeutic agents) with aptamers greatly prolongs the duration of therapeutic effect while reducing systemic toxicity (e.g., local anesthesia from site 1 sodium channel blockers). This approach provides drug delivery systems for a wide range of drugs and indications.

In particular, aptamers provide a sustained release carrier for drug delivery where extension of therapeutic effect is desirable and systemic toxicity from rapid release of drug can be problematic. In addition to improving duration of effect and limiting systemic toxicity, this new paradigm of drug delivery further provides a means of delivering hydrophilic compounds which are historically difficult to physically deliver via existing known methods of drug delivery (e.g., encapsulation).

In one aspect, provided is a composition comprising an aptamer and a molecular payload, wherein the molecular payload is bound to the aptamer, and wherein the aptamer is a sustained release carrier for the molecular payload. In certain embodiments, the composition is a depot for the molecular payload.

### Aptamer

As described herein, the aptamer is a sustained release carrier. In certain embodiments, the sustained release carrier facilitates local sustained release of the molecular payload. In certain embodiments, the sustained release carrier facilitates systemic sustained release of the molecular payload. In certain embodiments, the aptamer specifically binds to the molecular payload.

In certain embodiments, the aptamer is an oligonucleotide. In certain embodiments, the aptamer is a single stranded oligonucleotide. In certain embodiments, the aptamer is a deoxyribonucleic acid (DNA) or a ribonucleic acid (RNA). In certain embodiments, the aptamer is a deoxyribonucleic acid (DNA). In certain embodiments, the aptamer is a ribonucleic acid (RNA).

The aptamer oligonucleotide may include one or more modified nucleotides. In certain embodiments, a modified nucleotide includes a 2′-modified nucleotide (i.e., a nucleotide with a group other than a hydroxyl group at the 2′ position of the five-membered sugar ring). Modified nucleotides include, but are not limited to: 2′-modified nucleotides, 2′-O-methyl nucleotides (represented herein as a lower case letter ‘m’ in a nucleotide sequence), 2′-deoxy-2′-fluoro nucleotides (represented herein as a lower case letter ‘f’ in a nucleotide sequence), 2′-deoxy nucleotides, 2′-methoxyethyl (2′-O-2-methoxylethyl) nucleotides, 2′-amino nucleotides, 2′-alkyl nucleotides, 3′ to 3′ linkages (inverted) nucleotides, non-natural base including nucleotides, locked nucleotides, bridged nucleotides, peptide nucleic acids, 2′,3′-seco nucleotide mimics (unlocked nucleobase analogues), locked nucleotide, 3′-O-methoxy (2′ internucleotide linked) nucleotide, 2′-F-arabino nucleotides, morpholino nucleotides, vinyl phosphonate deoxyribonucleotide, vinyl phosphonate nucleotides, and abasic nucleotides. It is not necessary for all positions in a given compound to be uniformly modified. Conversely, more than one modification may be incorporated in a single oligomer or even in a single nucleotide thereof. The oligonucleotide may be synthesized and/or modified by methods known in the art. Modification at each nucleotide is independent of modification of the other nucleotides.

Modified nucleobases include synthetic and natural nucleobases, such as 5-substituted pyrimidines, 6-azapyrimidines, N-2-, N-6-, and O-6-substituted purines (e.g., 2-aminopropyladenine), 5-propynyluracil, 5-propynylcytosine, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine, 2-thiocytosine, 5-halouracil, 5-halocytosine, 5-propynyl uracil, 5-propynyl cytosine, 6-azo-uracil, 6-azo-cytosine, 6-azo-thymine, 5-uracil (pseudouracil), 4-thiouracil, 8-halo, 8-amino, 8-thiol, 8-thioalkyl, 8-hydroxyl and other 8-substituted adenines and guanines, 5-substituted uracils and cytosines (e.g., 5-halo uracils and cytosines (e.g., 5-bromouracil and 5-bromocytosine), 5-trifluoromethyl uracil, 5-trifluoromethyl cytosine), 7-methylguanine, 7-methyladenine, 8-azaguanine, 8-azaadenine, 7-deazaguanine, 7-deazaadenine, 3-deazaguanine, and 3-deazaadenine.

In certain embodiments, the aptamer is an oligonucleotide sequence of 10-200, 10-150, 10-100, 10-60, 15-60, 15-40, 15-35, 20-35, 25-35, 30-35, or 30-34 nucleotides. In certain embodiments, the aptamer is an oligonucleotide sequence of 15-60 nucleotides. In certain embodiments, the aptamer is an oligonucleotide sequence of 15-40 nucleotides. In certain embodiments, the aptamer is an oligonucleotide sequence of 25-35 nucleotides. In certain embodiments, the aptamer is an oligonucleotide sequence of 30-35 nucleotides.

In certain embodiments, the aptamer is an oligonucleotide sequence of about 10, about 15, about 20, about 25, about 30, about 35, about 40, about 45, about 50, about 55, or about 60 nucleotides. In certain embodiments, the aptamer is an oligonucleotide sequence of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides. In certain embodiments, the aptamer is an oligonucleotide sequence of 21 nucleotides. In certain embodiments, the aptamer is an oligonucleotide sequence of 30 nucleotides. In certain embodiments, the aptamer is an oligonucleotide sequence of 34 nucleotides. In certain embodiments, the aptamer is an oligonucleotide sequence of 44 nucleotides.

For the oligonucleotide, any nucleotides may be linked by phosphate-containing or non-phosphate-containing covalent internucleoside or internucleotide linkages. Modified internucleoside or internucleotide linkages or backbones include, but are not limited to, phosphorothioate group (represented herein as an asterisk (*) or a lower case ‘s’ after a nucleotide, as in As, mUs, fAx), chiral phosphorothioates, thiophosphate, phosphorodithioates, phosphotriesters, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkyl-phosphonates, thionoalkylphosphotriesters, morpholino linkages, boranophosphates having normal 3′-5′ linkages, 2′-5′ linked analogs of boranophosphates, and boranophosphates having inverted polarity wherein the adjacent pairs of nucleoside units are linked 3′-5′ to 5′-3′ or 2′-5′ to 5′-2′. In some embodiments, a modified internucleoside or internucleotide linkage or backbone lacks a phosphorus atom. Modified internucleoside or internucleotide linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside or internucleotide backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In certain embodiments, the aptamer is an oligonucleotide sequence having at least one phosphorothioate internucleotide linkage. In certain embodiments, the aptamer is an oligonucleotide sequence wherein all of the internucleotide linkages are phosphorothioate internucleotide linkages. In certain embodiments, the aptamer is an oligonucleotide sequence having a complete phosphorothioate backbone.

In certain embodiments, the aptamer is a single-stranded deoxyribonucleic acid (DNA) having a complete phosphorothioate backbone. In certain embodiments, the aptamer is a single-stranded deoxyribonucleic acid (DNA) of 15-60 nucleotides having a complete phosphorothioate backbone. In certain embodiments, the aptamer is a single-stranded deoxyribonucleic acid (DNA) of 15-60 nucleotides having a complete phosphorothioate backbone. In certain embodiments, the aptamer is a single-stranded deoxyribonucleic acid (DNA) of 15-40 nucleotides having a complete phosphorothioate backbone. In certain embodiments, the aptamer is a single-stranded deoxyribonucleic acid (DNA) of 25-35 nucleotides having a complete phosphorothioate backbone. In certain embodiments, the aptamer is a single-stranded deoxyribonucleic acid (DNA) of 30-35 nucleotides having a complete phosphorothioate backbone. In certain embodiments, the aptamer is a single-stranded deoxyribonucleic acid (DNA) of about 30 nucleotides having a complete phosphorothioate backbone. In certain embodiments, the aptamer is a single-stranded deoxyribonucleic acid (DNA) of about 34 nucleotides having a complete phosphorothioate backbone.

In certain embodiments, the nucleotide sequence of the aptamer has at least 85%, 86%, 87%, 88%, 89%, 90%, 91%, 92%, 93%, 94%, 95%, 96%, 97%, 98%, 99%, or 99.5% sequence identity to the nucleotide sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

In certain embodiments, the nucleotide sequence of the aptamer has at least 85%, 86%, 87%, 88%, 89%, 90%, 91%, 92%, 93%, 94%, 95%, 96%, 97%, 98%, 99%, or 99.5% sequence identity to the nucleotide sequence of SEQ ID NO: 1, SEQ ID NO: 2, SEQ ID NO: 3, SEQ ID NO: 4, or SEQ ID NO: 5.

In certain embodiments, the nucleotide sequence of the aptamer has at least 85%, 86%, 87%, 88%, 89%, 90%, 91%, 92%, 93%, 94%, 95%, 96%, 97%, 98%, 99%, or 99.5% sequence identity to the nucleotide sequence of SEQ ID NO: 1, SEQ ID NO: 2, or SEQ ID NO: 5.

In certain embodiments, the nucleotide sequence of the aptamer has at least 85%, 86%, 87%, 88%, 89%, 90%, 91%, 92%, 93%, 94%, 95%, 96%, 97%, 98%, 99%, or 99.5% sequence identity to the nucleotide sequence of SEQ ID NO: 2 or SEQ ID NO: 5.

In certain embodiments, the nucleotide sequence of the aptamer has at least 85%, 86%, 87%, 88%, 89%, 90%, 91%, 92%, 93%, 94%, 95%, 96%, 97%, 98%, 99%, or 99.5% sequence identity to the nucleotide sequence of SEQ ID NO: 1.

In certain embodiments, the nucleotide sequence of the aptamer has at least 85%, 86%, 87%, 88%, 89%, 90%, 91%, 92%, 93%, 94%, 95%, 96%, 97%, 98%, 99%, or 99.5% sequence identity to the nucleotide sequence of SEQ ID NO: 2.

In certain embodiments, the nucleotide sequence of the aptamer has at least 85%, 86%, 87%, 88%, 89%, 90%, 91%, 92%, 93%, 94%, 95%, 96%, 97%, 98%, 99%, or 99.5% sequence identity to the nucleotide sequence of SEQ ID NO: 3.

In certain embodiments, the nucleotide sequence of the aptamer has at least 85%, 86%, 87%, 88%, 89%, 90%, 91%, 92%, 93%, 94%, 95%, 96%, 97%, 98%, 99%, or 99.5% sequence identity to the nucleotide sequence of SEQ ID NO: 4.

In certain embodiments, the nucleotide sequence of the aptamer has at least 85%, 86%, 87%, 88%, 89%, 90%, 91%, 92%, 93%, 94%, 95%, 96%, 97%, 98%, 99%, or 99.5% sequence identity to the nucleotide sequence of SEQ ID NO: 5.

In certain embodiments, the nucleotide sequence of the aptamer has at least 85%, 86%, 87%, 88%, 89%, 90%, 91%, 92%, 93%, 94%, 95%, 96%, 97%, 98%, 99%, or 99.5% sequence identity to the nucleotide sequence of SEQ ID NO: 6.

In certain embodiments, the nucleotide sequence of the aptamer has at least 85%, 86%, 87%, 88%, 89%, 90%, 91%, 92%, 93%, 94%, 95%, 96%, 97%, 98%, 99%, or 99.5% sequence identity to the nucleotide sequence of SEQ ID NO: 7.

In certain embodiments, the nucleotide sequence of the aptamer has at least 85%, 86%, 87%, 88%, 89%, 90%, 91%, 92%, 93%, 94%, 95%, 96%, 97%, 98%, 99%, or 99.5% sequence identity to the nucleotide sequence of SEQ ID NO: 8.

In certain embodiments, the nucleotide sequence of the aptamer has at least 85%, 86%, 87%, 88%, 89%, 90%, 91%, 92%, 93%, 94%, 95%, 96%, 97%, 98%, 99%, or 99.5% sequence identity to the nucleotide sequence of SEQ ID NO: 9.

In certain embodiments, the nucleotide sequence of the aptamer has at least 85%, 86%, 87%, 88%, 89%, 90%, 91%, 92%, 93%, 94%, 95%, 96%, 97%, 98%, 99%, or 99.5% sequence identity to the nucleotide sequence of SEQ ID NO: 10.

In certain embodiments, the nucleotide sequence of the aptamer has at least 85%, 86%, 87%, 88%, 89%, 90%, 91%, 92%, 93%, 94%, 95%, 96%, 97%, 98%, 99%, or 99.5% sequence identity to the nucleotide sequence of SEQ ID NO: 11.

In certain embodiments, the aptamer is SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11. In certain embodiments, the aptamer is SEQ ID NO: 1, SEQ ID NO: 2, SEQ ID NO: 3, SEQ ID NO: 4, or SEQ ID NO: 5. In certain embodiments, the aptamer is SEQ ID NO: 1, SEQ ID NO: 2, or SEQ ID NO: 5. In certain embodiments, the aptamer is SEQ ID NO: 2 or SEQ ID NO: 5. In certain embodiments, the aptamer is SEQ ID NO: 1. In certain embodiments, the aptamer is SEQ ID NO: 2. In certain embodiments, the aptamer is SEQ ID NO: 3. In certain embodiments, the aptamer is SEQ ID NO: 4. In certain embodiments, the aptamer is SEQ ID NO: 5. In certain embodiments, the aptamer is SEQ ID NO: 6. In certain embodiments, the aptamer is SEQ ID NO: 7. In certain embodiments, the aptamer is SEQ ID NO: 8. In certain embodiments, the aptamer is SEQ ID NO: 9. In certain embodiments, the aptamer is SEQ ID NO: 10. In certain embodiments, the aptamer is SEQ ID NO: 11.

In certain embodiments, the aptamer is not a targeting moiety (i.e., it does not bind to a biological target of interest). In certain embodiments, the aptamer is not a therapeutic agent. In certain embodiments, the aptamer is a therapeutic agent, but does not function as a therapeutic agent in the composition.

### Molecular Payload

In certain embodiments, the molecular payload is a therapeutic moiety or therapeutic agent. In certain embodiments, the molecular payload is a therapeutic agent. In certain embodiments, the molecular payload is a biologic or a small molecule. In certain embodiments, the molecular payload is a combination of a biologic and a small molecule (e.g., and antibody-drug conjugate). In certain embodiments, the molecular payload is a polynucleotide, a polypeptide, or a small molecule. In certain embodiments, the molecular payload is a small molecule. In certain embodiments, the molecular payload is a biologic.

In certain embodiments, the molecular payload is an anesthetic, an antibiotic, or a neurotransmitter.

In certain embodiments, the molecular payload is an anesthetic. In certain embodiments, the molecular payload is a site 1 sodium channel blocker. In certain embodiments, the molecular payload is tetrodotoxin or saxitoxin. In certain embodiments, the molecular payload is tetrodotoxin. In certain embodiments, the molecular payload is saxitoxin.

In certain embodiments, the molecular payload is an antibiotic. In certain embodiments, the molecular payload is an aminoglycoside antibiotic. In certain embodiments, the molecular payload is kanamycin. In certain embodiments, the molecular payload is a kanamycin analog.

In certain embodiments, the molecular payload is a neurotransmitter. In certain embodiments, the molecular payload is serotonin. In certain embodiments, the molecular payload is a serotonin analog.

In certain embodiments, the molecular payload is a polypeptide. In certain embodiments, the molecular payload is a peptide hormone. In certain embodiments, the molecular payload is insulin. In certain embodiments, the molecular payload is synthetic insulin. In certain embodiments, the molecular payload is an insulin analog.

In certain embodiments, the molecular payload is not covalently bound to the aptamer.

In certain embodiments, the molecular payload is hydrophilic. In certain embodiments, the molecular payload is hydrophobic. In certain embodiments, the molecular payload has a Log D or Log P that is equal to or less than 0. In certain embodiments, the molecular payload has a Log D or Log P that is equal to or less than 0.5. In certain embodiments, the molecular payload has a Log D or Log P that is equal to or less than 1. In certain embodiments, the molecular payload has a Log D or Log P that is equal to or less than 1.5. In certain embodiments, the molecular payload has a Log D or Log P that is equal to or less than 2. In certain embodiments, the molecular payload has a Log D or Log P that is equal to or less than 2.5. In certain embodiments, the molecular payload has a Log D or Log P that is equal to or less than 3. In certain embodiments, the molecular payload has a Log D or Log P that is equal to or less than 3.5. In certain embodiments, the molecular payload has a Log D or Log P that is equal to or less than 4. In certain embodiments, the molecular payload has a Log D or Log P that is equal to or less than 4.5. In certain embodiments, the molecular payload has a Log D or Log P that is equal to or less than 5. In certain embodiments, the molecular payload has a Log D or Log P that is equal to or less than −0.5. In certain embodiments, the molecular payload has a Log D or Log P that is equal to or less than −1. In certain embodiments, the molecular payload has a Log D or Log P that is equal to or less than −1.5. In certain embodiments, the molecular payload has a Log D or Log P that is equal to or less than −2. In certain embodiments, the molecular payload has a Log D or Log P that is equal to or less than −2.5. In certain embodiments, the molecular payload has a Log D or Log P that is equal to or less than −3. In certain embodiments, the molecular payload has a Log D or Log P that is equal to or less than −3.5. In certain embodiments, the molecular payload has a Log D or Log P that is equal to or less than −4. In certain embodiments, the molecular payload has a Log D or Log P that is equal to or less than −4.5. In certain embodiments, the molecular payload has a Log D or Log P that is equal to or less than −5. In certain embodiments, the molecular payload has a Log D or Log P that is −5 to 0. In certain embodiments, the molecular payload has a Log D or Log P that is 0 to 5.

In certain embodiments, the composition comprises an effective amount of the molecular payload. In certain embodiments, the composition comprises a therapeutically effective amount of the molecular payload. In certain embodiments, the composition comprises a prophylactically effective amount of the molecular payload. In certain embodiments, the effective amount is an amount effective for treating a disease or disorder that would benefit from administration of a therapeutic agent in a subject in need thereof. In certain embodiments, the effective amount is an amount effective for preventing a disease or disorder that would benefit from administration of a therapeutic agent in a subject in need thereof. In certain embodiments, the effective amount is an amount effective for reducing the risk of developing a disease or disorder that would benefit from administration of a therapeutic agent in a subject in need thereof.

### Embodiments of the Composition

In certain embodiments, the molar ratio of aptamer to molecular payload is greater than 1.0. In certain embodiments, the molar ratio of aptamer to molecular payload is greater than 1.0, greater than 2.0, greater than 3.0, greater than 4.0, greater than 5.0, greater than 10.0, greater than 15.0, greater than 20.0, greater than 25.0, greater than 30.0, greater than 35.0, or greater than 40.0.

In certain embodiments, the molar ratio of aptamer to molecular payload is at least 1.0, at least 2.0, at least 3.0, at least 4.0, at least 5.0, at least 10.0, at least 15.0, at least 20.0, at least 25.0, at least 30.0, at least 35.0, or at least 40.0. In certain embodiments, the molar ratio of aptamer to molecular payload is at least 2.0, at least 3.0, at least 4.0, at least 5.0, at least 10.0, at least 15.0, at least 20.0, at least 25.0, at least 30.0, at least 35.0, or at least 40.0. In certain embodiments, the molar ratio of aptamer to molecular payload is at least 2.0. In certain embodiments, the molar ratio of aptamer to molecular payload is at least 3.0. In certain embodiments, the molar ratio of aptamer to molecular payload is at least 4.0. In certain embodiments, the molar ratio of aptamer to molecular payload is at least 5.0. In certain embodiments, the molar ratio of aptamer to molecular payload is at least 10.0. In certain embodiments, the molar ratio of aptamer to molecular payload is at least 15.0. In certain embodiments, the molar ratio of aptamer to molecular payload is at least 20.0. In certain embodiments, the molar ratio of aptamer to molecular payload is at least 25.0. In certain embodiments, the molar ratio of aptamer to molecular payload is at least 30.0. In certain embodiments, the molar ratio of aptamer to molecular payload is at least 35.0. In certain embodiments, the molar ratio of aptamer to molecular payload is at least 40.0.

In certain embodiments, the molar ratio of aptamer to molecular payload is about 2.0, about 3.0, about 4.0, about 5.0, about 10.0, about 15.0, about 20.0, about 25.0, about 30.0, about 35.0, or about 40.0. In certain embodiments, the molar ratio of aptamer to molecular payload is about 2.0. In certain embodiments, the molar ratio of aptamer to molecular payload is about 3.0. In certain embodiments, the molar ratio of aptamer to molecular payload is about 4.0. In certain embodiments, the molar ratio of aptamer to molecular payload is about 5.0. In certain embodiments, the molar ratio of aptamer to molecular payload is about 10.0. In certain embodiments, the molar ratio of aptamer to molecular payload is about 15.0. In certain embodiments, the molar ratio of aptamer to molecular payload is about 20.0. In certain embodiments, the molar ratio of aptamer to molecular payload is about 25.0. In certain embodiments, the molar ratio of aptamer to molecular payload is about 30.0. In certain embodiments, the molar ratio of aptamer to molecular payload is about 35.0. In certain embodiments, the molar ratio of aptamer to molecular payload is about 40.0.

In certain embodiments, the molar ratio of aptamer to molecular payload is from 1.0-20.0, 1.0-40.0, 1.1-20.0, 1.1-40.0, 2.0-20.0, or 2.0-40.0. In certain embodiments, the molar ratio of aptamer to molecular payload is from 1.0-20.0. In certain embodiments, the molar ratio of aptamer to molecular payload is from 1.0-40.0. In certain embodiments, the molar ratio of aptamer to molecular payload is from 1.1-20.0. In certain embodiments, the molar ratio of aptamer to molecular payload is from 1.1-40.0. In certain embodiments, the molar ratio of aptamer to molecular payload is from 2.0-20.0. In certain embodiments, the molar ratio of aptamer to molecular payload is from 2.0-40.0.

In certain embodiments, the composition is characterized in that, when tested in vitro by placing the composition in a medium, less than 100%, less than 90%, less than 80%, less than 70%, less than 60%, less than 50%, less than 40%, less than 30%, less than 20%, or less than 10% of the molecular payload is released from the composition 48 hours after placing the composition in the medium. In certain embodiments, the composition is characterized in that, when tested in vitro by placing the composition in a medium, less than 100%, less than 90%, less than 80%, less than 70%, less than 60%, less than 50%, less than 40%, less than 30%, less than 20%, or less than 10% of the molecular payload is released from the composition 24 hours after placing the composition in the medium. In certain embodiments, the composition is characterized in that, when tested in vitro by placing the composition in a medium, less than 100%, less than 90%, less than 80%, less than 70%, less than 60%, less than 50%, less than 40%, less than 30%, less than 20%, or less than 10% of the molecular payload is released from the composition 12 hours after placing the composition in the medium. In certain embodiments, the composition is characterized in that, when tested in vitro by placing the composition in a medium, less than 100%, less than 90%, less than 80%, less than 70%, less than 60%, less than 50%, less than 40%, less than 30%, less than 20%, or less than 10% of the molecular payload is released from the composition 6 hours after placing the composition in the medium. In certain embodiments, the composition is characterized in that, when tested in vitro by placing the composition in a medium, less than 100%, less than 90%, less than 80%, less than 70%, less than 60%, less than 50%, less than 40%, less than 30%, less than 20%, or less than 10% of the molecular payload is released from the composition 3 hours after placing the composition in the medium. In certain embodiments, the medium is phosphate-buffered saline (PBS) having a pH of about 7.4

In certain embodiments, the composition is characterized in that, when administered to a subject, less than 100%, less than 90%, less than 80%, less than 70%, less than 60%, less than 50%, less than 40%, less than 30%, less than 20%, or less than 10% of the molecular payload is released from the composition 48 hours after administration of the composition. In certain embodiments, the composition is characterized in that, when administered to a subject, less than 100%, less than 90%, less than 80%, less than 70%, less than 60%, less than 50%, less than 40%, less than 30%, less than 20%, or less than 10% of the molecular payload is released from the composition 24 hours after administration of the composition. In certain embodiments, the composition is characterized in that, when administered to a subject, less than 100%, less than 90%, less than 80%, less than 70%, less than 60%, less than 50%, less than 40%, less than 30%, less than 20%, or less than 10% of the molecular payload is released from the composition 12 hours after administration of the composition. In certain embodiments, the composition is characterized in that, when administered to a subject, less than 100%, less than 90%, less than 80%, less than 70%, less than 60%, less than 50%, less than 40%, less than 30%, less than 20%, or less than 10% of the molecular payload is released from the composition 6 hours after administration of the composition. In certain embodiments, the composition is characterized in that, when administered to a subject, less than 100%, less than 90%, less than 80%, less than 70%, less than 60%, less than 50%, less than 40%, less than 30%, less than 20%, or less than 10% of the molecular payload is released from the composition 3 hours after administration of the composition.

In certain embodiments, the composition is characterized in that, when administered to a subject, duration of a therapeutic effect is extended by at least 10%, at least 20%, at least 30%, at least 40%, at least 50%, at least 60%, at least 70%, at least 80%, at least 90%, at least 100%, or at least 200% compared to duration of the therapeutic effect upon administration of the molecular payload alone or in a composition without the aptamer. In certain embodiments, the therapeutic effect is nerve block and/or reduced pain. In certain embodiments, the therapeutic effect is nerve block. In certain embodiments, the therapeutic effect is reduced pain.

The present disclosure also provides methods for preparing the compositions disclosed herein. In certain embodiments, the method comprises providing a molecular payload; identifying or preparing an aptamer that binds to the molecular payload; and combining the molecular payload and aptamer in a composition. In certain embodiments, the molecular payload is any molecular payload as defined herein. In certain embodiments, the aptamer is any aptamer as defined herein. The aptamer and molecular payload may be combined in any manner known by one of ordinary skill in the art.

### Pharmaceutical Compositions, Kits, and Administration

The present disclosure provides compositions comprising an aptamer and a molecular payload. In certain embodiments, the composition is a pharmaceutical composition. In certain embodiments, the composition further comprises a pharmaceutically acceptable excipient.

In certain embodiments, the pharmaceutical composition comprises an effective amount of a molecular payload. In certain embodiments, the effective amount is a therapeutically effective amount. In certain embodiments, the effective amount is a prophylactically effective amount. In certain embodiments, the effective amount is an amount effective for treating a disease or condition in a subject in need thereof. In certain embodiments, the effective amount is an amount effective for preventing a disease or condition in a subject in need thereof. In certain embodiments, the effective amount is an amount effective for reducing the risk of developing a disease or condition in a subject in need thereof. In certain embodiments, the molecular payload is a therapeutic agent.

Compositions described herein can be prepared by any method known in the art of pharmacology. In general, such preparatory methods include the steps of bringing the composition comprising a molecular payload (e.g., a therapeutic agent) into association with an aptamer. In certain embodiments, the preparation further comprises adding and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

The compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical, mucosal, nasal, bucal, sublingual; by intratracheal instillation, bronchial instillation, and/or inhalation; and/or as an oral spray, nasal spray, and/or aerosol.

A composition, as described herein, can be administered in combination with one or more additional agents (e.g., pharmaceutical agent, e.g., therapeutically and/or prophylactically active agents). The compositions can be administered in combination with additional pharmaceutical agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve their ability to cross the blood-brain barrier, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a therapeutic agent described herein and an additional agent exhibits a synergistic effect that is absent in a pharmaceutical composition including one of the therapeutic agent and the additional agent, but not both. In certain embodiments, the additional agent is a therapeutic agent. In certain embodiments, the additional agent is epinephrine.

The composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

Also encompassed by the disclosure are kits (e.g., pharmaceutical packs). The kits provided may comprise a composition described herein and a container (e.g., a vial, ampule, bottle, syringe, and/or dispenser package, or other suitable container). In some embodiments, provided kits may optionally further include a second container comprising a pharmaceutical excipient for dilution or suspension of a composition described herein. In some embodiments, the composition described herein provided in the first container and the second container are combined to form one unit dosage form.

Thus, in one aspect, provided are kits including a first container comprising a composition described herein. In certain embodiments, the kits are useful for treating a disease or condition (e.g., pain) in a subject in need thereof. In certain embodiments, the kits are useful for preventing a disease or condition (e.g., pain) in a subject in need thereof. In certain embodiments, the kits are useful for reducing the risk of developing a disease or condition (e.g., pain) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, a kit described herein may include one or more additional pharmaceutical agents described herein as a separate composition.

### Methods of Use

The present disclosure also provides methods for treating diseases or conditions in a subject in need thereof, the methods comprising administering to the subject a provided composition. The present disclosure also provides methods for preventing diseases or conditions in a subject in need thereof, the methods comprising administering to the subject a provided composition. The present disclosure also provides methods for reducing the risk of developing a disease or condition in a subject in need thereof.

In certain embodiments, the disclosed compositions can be used to treat a subject (e.g., a human) having a disease or condition that would benefit from administration of a molecular payload (e.g., a therapeutic agent). In certain embodiments, the subject is administered an effective amount of any one or more disclosed compositions. In certain embodiments, the subject is administered a therapeutically effective amount of any one or more disclosed compositions. In certain embodiments, the subject is administered a prophylactically effective amount of any one or more disclosed compositions. In certain embodiments, the subject is an animal. In certain embodiments, the subject is a mammal. In certain embodiments, the subject is a human. In certain embodiments, the subject is a human aged 18 years or older. In certain embodiments, the subject is a human aged 12-18 years, exclusive. In certain embodiments, the subject is a human aged 2-12 years, inclusive. In certain embodiments, the subject is a human younger than 2 years. In certain embodiments, the subject is a non-human animal. In certain embodiments, the subject is a non-human mammal.

In certain embodiments, the disease or condition being treated is one associated with a specific biological target. In certain embodiments, the target is a voltage-gated sodium channel. In certain embodiments, the target is a site-1 sodium channel. In certain embodiments, the disease or condition is pain.

In certain embodiments, the disease or condition is an infection. In certain embodiments, the disease or condition is a bacterial infection. In certain embodiments, the disease or condition is tuberculosis. In certain embodiments, the disease or condition is diabetes (e.g., type 1 diabetes, type 2 diabetes, gestational diabetes).

In certain embodiments, the methods for treating diseases or conditions comprise administering the composition such that the molecular payload (e.g., therapeutic agent) is delivered to a biological target at a rate that is less than if the molecular payload were administered alone. In such embodiments, the molecular payload is delivered via sustained release. In certain embodiments, the administration is local such that the sustained release is a local sustained release (i.e., local administration for a local effect, e.g., subcutaneous administration). In certain embodiments, administration is systemic such that the sustained release is a systemic sustained release (i.e., systemic administration for a dispersed or system-wide effect, e.g., parenteral administration).

In certain embodiments, less than 100%, less than 90%, less than 80%, less than 70%, less than 60%, less than 50%, less than 40%, less than 30%, less than 20%, or less than 10% of the molecular payload (e.g., therapeutic agent) is released from the composition 48 hours after administration of the composition to the subject. In certain embodiments, less than 100%, less than 90%, less than 80%, less than 70%, less than 60%, less than 50%, less than 40%, less than 30%, less than 20%, or less than 10% of the molecular payload (e.g., therapeutic agent) is released from the composition 24 hours after administration of the composition to the subject. In certain embodiments, less than 100%, less than 90%, less than 80%, less than 70%, less than 60%, less than 50%, less than 40%, less than 30%, less than 20%, or less than 10% of the molecular payload (e.g., therapeutic agent) is released from the composition 12 hours after administration of the composition to the subject. In certain embodiments, less than 100%, less than 90%, less than 80%, less than 70%, less than 60%, less than 50%, less than 40%, less than 30%, less than 20%, or less than 10% of the molecular payload (e.g., therapeutic agent) is released from the composition 6 hours after administration of the composition to the subject. In certain embodiments, less than 100%, less than 90%, less than 80%, less than 70%, less than 60%, less than 50%, less than 40%, less than 30%, less than 20%, or less than 10% of the molecular payload (e.g., therapeutic agent) is released from the composition 3 hours after administration of the composition to the subject. In certain embodiments, less than 100%, less than 90%, less than 80%, less than 70%, less than 60%, less than 50%, less than 40%, less than 30%, less than 20%, or less than 10% of the molecular payload (e.g., therapeutic agent) is released from the aptamer 48 hours after administration of the composition to the subject. In certain embodiments, less than 100%, less than 90%, less than 80%, less than 70%, less than 60%, less than 50%, less than 40%, less than 30%, less than 20%, or less than 10% of the molecular payload (e.g., therapeutic agent) is released from the aptamer 24 hours after administration of the composition to the subject. In certain embodiments, less than 100%, less than 90%, less than 80%, less than 70%, less than 60%, less than 50%, less than 40%, less than 30%, less than 20%, or less than 10% of the molecular payload (e.g., therapeutic agent) is released from the aptamer 12 hours after administration of the composition to the subject. In certain embodiments, less than 100%, less than 90%, less than 80%, less than 70%, less than 60%, less than 50%, less than 40%, less than 30%, less than 20%, or less than 10% of the molecular payload (e.g., therapeutic agent) is released from the aptamer 6 hours after administration of the composition to the subject. In certain embodiments, less than 100%, less than 90%, less than 80%, less than 70%, less than 60%, less than 50%, less than 40%, less than 30%, less than 20%, or less than 10% of the molecular payload (e.g., therapeutic agent) is released from the aptamer 3 hours after administration of the composition to the subject.

In certain embodiments, duration of a therapeutic effect is extended by at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1,000%, or at least 10,000% compared to duration of a therapeutic effect upon administration of the molecular payload (e.g., therapeutic agent) alone or in a composition without the aptamer. In certain embodiments, duration of a therapeutic effect is extended by at least 10% compared to duration of a therapeutic effect upon administration of the molecular payload (e.g., therapeutic agent) alone or in a composition without the aptamer. In certain embodiments, duration of a therapeutic effect is extended by at least 20% compared to duration of a therapeutic effect upon administration of the molecular payload (e.g., therapeutic agent) alone or in a composition without the aptamer. In certain embodiments, duration of a therapeutic effect is extended by at least 30% compared to duration of a therapeutic effect upon administration of the molecular payload (e.g., therapeutic agent) alone or in a composition without the aptamer. In certain embodiments, duration of a therapeutic effect is extended by at least 40% compared to duration of a therapeutic effect upon administration of the molecular payload (e.g., therapeutic agent) alone or in a composition without the aptamer. In certain embodiments, duration of a therapeutic effect is extended by at least 50% compared to duration of a therapeutic effect upon administration of the molecular payload (e.g., therapeutic agent) alone or in a composition without the aptamer. In certain embodiments, duration of a therapeutic effect is extended by at least 60% compared to duration of a therapeutic effect upon administration of the molecular payload (e.g., therapeutic agent) alone or in a composition without the aptamer. In certain embodiments, duration of a therapeutic effect is extended by at least 70% compared to duration of a therapeutic effect upon administration of the molecular payload (e.g., therapeutic agent) alone or in a composition without the aptamer. In certain embodiments, duration of a therapeutic effect is extended by at least 80% compared to duration of a therapeutic effect upon administration of the molecular payload (e.g., therapeutic agent) alone or in a composition without the aptamer. In certain embodiments, duration of a therapeutic effect is extended by at least 90% compared to duration of a therapeutic effect upon administration of the molecular payload (e.g., therapeutic agent) alone or in a composition without the aptamer. In certain embodiments, duration of a therapeutic effect is extended by at least 100% compared to duration of a therapeutic effect upon administration of the molecular payload (e.g., therapeutic agent) alone or in a composition without the aptamer. In certain embodiments, duration of a therapeutic effect is extended by least 200% compared to duration of a therapeutic effect upon administration of the molecular payload (e.g., therapeutic agent) alone or in a composition without the aptamer. In certain embodiments, duration of a therapeutic effect is extended by least 300% compared to duration of a therapeutic effect upon administration of the molecular payload (e.g., therapeutic agent) alone or in a composition without the aptamer. In certain embodiments, duration of a therapeutic effect is extended by least 400% compared to duration of a therapeutic effect upon administration of the molecular payload (e.g., therapeutic agent) alone or in a composition without the aptamer. In certain embodiments, duration of a therapeutic effect is extended by least 500% compared to duration of a therapeutic effect upon administration of the molecular payload (e.g., therapeutic agent) alone or in a composition without the aptamer. In certain embodiments, duration of a therapeutic effect is extended by least 600% compared to duration of a therapeutic effect upon administration of the molecular payload (e.g., therapeutic agent) alone or in a composition without the aptamer. In certain embodiments, duration of a therapeutic effect is extended by least 700% compared to duration of a therapeutic effect upon administration of the molecular payload (e.g., therapeutic agent) alone or in a composition without the aptamer. In certain embodiments, duration of a therapeutic effect is extended by least 800% compared to duration of a therapeutic effect upon administration of the molecular payload (e.g., therapeutic agent) alone or in a composition without the aptamer. In certain embodiments, duration of a therapeutic effect is extended by least 900% compared to duration of a therapeutic effect upon administration of the molecular payload (e.g., therapeutic agent) alone or in a composition without the aptamer. In certain embodiments, duration of a therapeutic effect is extended by least 1,000% compared to duration of a therapeutic effect upon administration of the molecular payload (e.g., therapeutic agent) alone or in a composition without the aptamer. In certain embodiments, duration of a therapeutic effect is extended by least 10,000% compared to duration of a therapeutic effect upon administration of the molecular payload (e.g., therapeutic agent) alone or in a composition without the aptamer.

In certain embodiments, the therapeutic effect is nerve block and/or reduced pain. In certain embodiments, the therapeutic effect is nerve block or reduced pain. In certain embodiments, the therapeutic effect is nerve block and reduced pain. In certain embodiments, the therapeutic effect is nerve block. In certain embodiments, the therapeutic effect is reduced pain. In certain embodiments, the therapeutic effect is reduction of pain by at least 10%, at least 20%, at least 30%, at least 40%, at least 50%, at least 60%, at least 70%, at least 80%, at least 90%, or at least 99% compared to a baseline level of pain prior to administration.

The present disclosure also provides uses of a provided composition in a method described herein. The present disclosure also provides uses of a provided pharmaceutical composition in a method described herein. The present disclosure also provides a provided composition for use in a method described herein. The present disclosure also provides a provided pharmaceutical composition for use in a method described herein.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Example 1. Design of Chemical Modified Aptamer for TTX Binding and Control Release

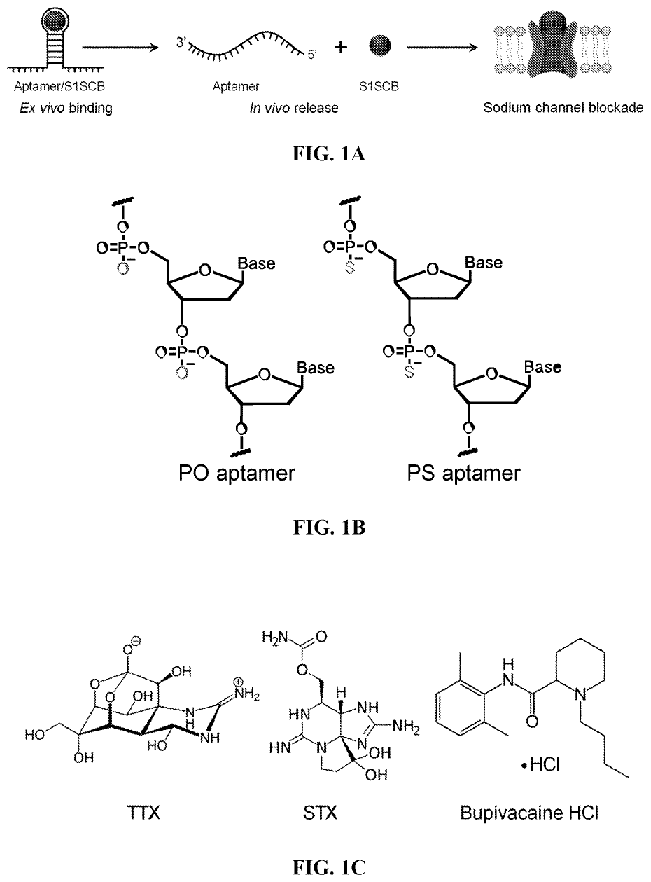

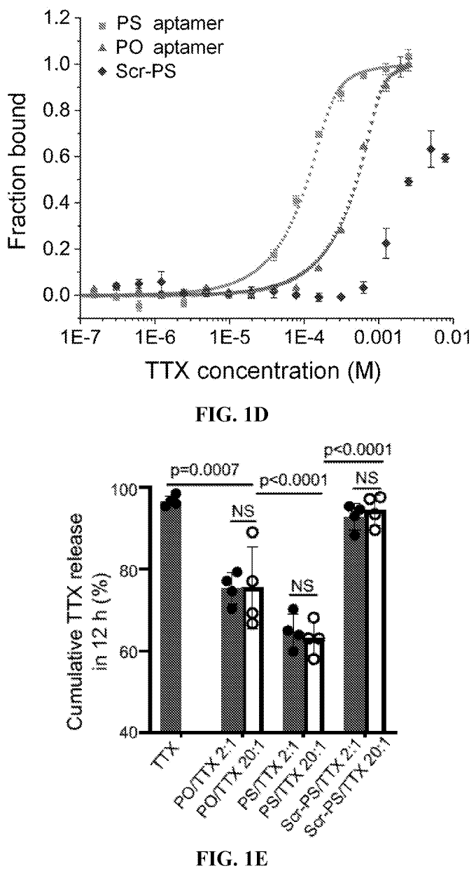

A high-affinity TTX-binding aptamer (5′-AAAAATTTCACACGGGTGCCTCGGCTGTCC-3′ (SEQ ID NO: 1)) was prepared to create a non-covalent complex with TTX (FIG. 1A and Table 1). To enhance resistance of the aptamer to nucleases, the phosphodiester (PO) backbone of was chemically modified with phosphorothioate (PS) (FIG. 1B and Table 1).

To assess the binding affinity of TTX (FIG. 1C) to the PS aptamer, interactions between TTX (in PBS) and a Cy5-labeled aptamer (Table 1) were studied by microscale thermophoresis (MST), allowing determination of K_{d} values. PS modification improved the binding affinity of aptamer to TTX by a factor of 2, from a K_{d} of 29.5±2.1 μM for the unmodified PO aptamer to 14.3±1.7 μM (FIG. 1D). A PS aptamer containing a scrambled sequence (Scr-PS) (Table 1) exhibited decreased binding with TTX (K_{d}=3.84 mM).

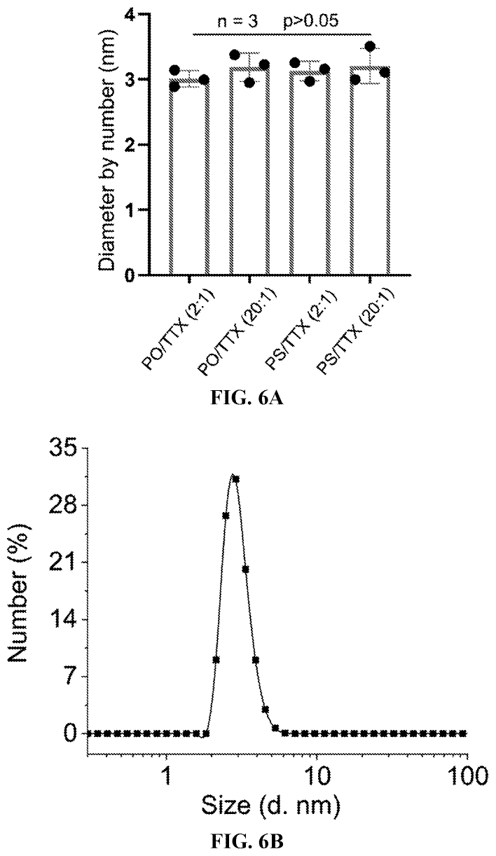

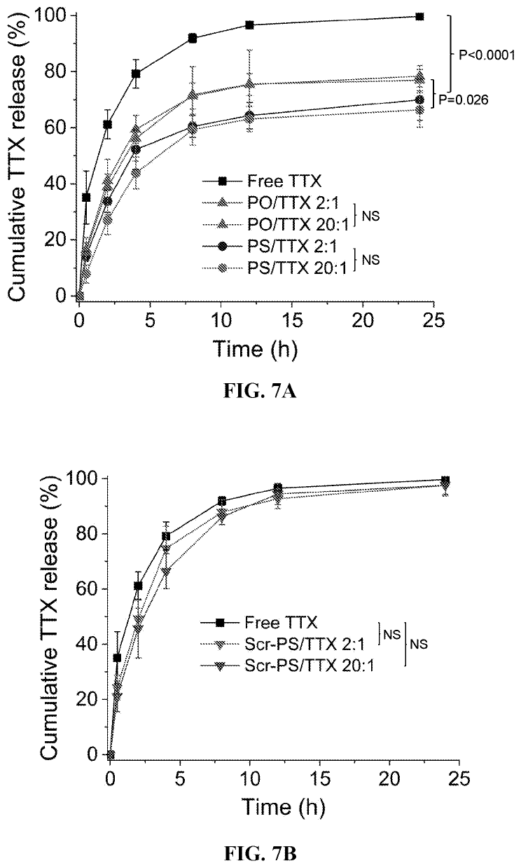

To test if this binding affinity could be the basis of a sustained release system, TTX was complexed with aptamers (PO or PS) in a 2:1 or 20:1 molar ratio (aptamer:TTX) by simple mixing, and release kinetics of TTX from these formulations were investigated. These samples exhibited very similar mean diameters ~3 nm measured by dynamic light scattering (DLS) (FIGS. 6A-6B), indicating that association with TTX did not cause aggregation of aptamers. The release kinetics of TTX were studied by dialyzing 200 μL of aptamer/TTX (the concentration of TTX was 42 μM; the aptamer concentration varied) against 14 mL of PBS. The release of TTX was quantified by enzyme-linked immunosorbent assay (ELISA). Both PO/TTX and PS/TTX complexes (2:1 or 20:1) increased the duration of TTX release compared to free TTX (p<0.0001 at 24 h, FIG. 1E and FIGS. 7A-7B). Release from PS/TTX was statistically significantly slower than from PO/TTX at both 12 h (p<0.0001) and 24 h (p=0.026). There was no statistically significant difference in TTX release between the two molar ratios tested (2:1, 20:1), for PO/TTX or PS/TTX (p>0.05). To verify that the sequence is essential for aptamer interactions with TTX, the release kinetics of the non-selective scrambled PS aptamer (Scr-PS; Table 1) affixed to TTX (Scr-PS/TTX) in two different molar ratios 2:1 and 20:1 were evaluated. Release of TTX from Scr-PS/TTX was more rapid than from PS/TTX, with 92.8±3.6% (2:1) and 94.4±3.8% (20:1) of TTX released in 12 h, which were similar to the rate of release of TTX without aptamer (p>0.05 for all comparisons). These data demonstrate that the interaction between aptamer and TTX was sequence specific, and that the PS modification slowed release compared to PO. The slower release from PS/TTX is attributed to the improved binding affinity between the PS aptamer and TTX.

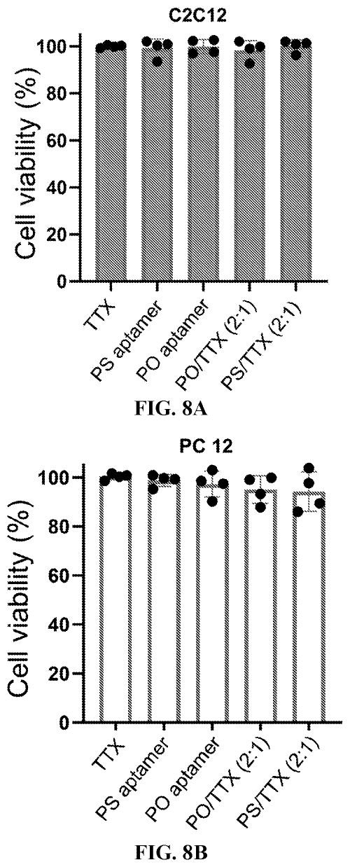

The cytotoxicity of aptamer/TTX was tested in two cell lines relevant to local anesthetic-related tissue injury: the myoblast C2C12 cell line was used to assess potential myotoxicity and the pheochromocytoma PC12 cell line was used to assess potential neurotoxicity. Cells were incubated with free TTX, PO aptamer, PS aptamer, PO/TTX (2:1) complex, or PS/TTX (2:1) complex at the same TTX concentration of 73 μM and/or the aptamer concentration of 146 μM for 24 hours and the cell viability was measured with the 3-(4,5-dimethylthiazol-2-yl)-5-(3-carboxymethoxyphenyl)-2-(4-sulfophenyl)-2H-tetrazolium) (MTS) assay. All groups in both cell types-maintained cell viability >90% compared with untreated cells (FIGS. 8A-8B).

### Example 2. Comparing the Effectiveness of Free TTX and Aptamer/TTX Complexes on Rat Sciatic Nerve Blockade In Vivo

To explore the feasibility of aptamers as a drug delivery system in vivo, the ability of aptamers to extend the duration of rat sciatic nerve block was investigated. Male Sprague-Dawley rats (n=4 or 6 per group) were injected at the left sciatic nerve with 0.3 mL of TTX alone or combined with an aptamer (PO, PS, or Scr-PS)/TTX. As 4 μg (42 μM) of free TTX in PBS provides nerve block without lethality in rats, it was set as the initial TTX dose for all formulations. The aptamer (PO or PS)/TTX complexes were first tested at a fixed aptamer:TTX molar ratio of 20:1. After injection, the rats underwent neurobehavioral testing to determine the duration of functional deficits (sensory and motor nerve blockade) in both hind paws. Thermal latency (the time in seconds that the rat leaves its hindpaw on the hotplate) was the primary metric for sensory nerve block. A sensory nerve block was considered successful if the latency was longer than 7 s. The duration of the sensory nerve block was defined as the time for thermal latency to return to 7 s (the midpoint between baseline (2 s) and maximal latency (12 s)). Deficits in the left (ipsilateral) hindpaw reflect nerve block, while deficits in the right (contralateral) hindpaw reflect systemically distributed drug (i.e., systemic toxicity).

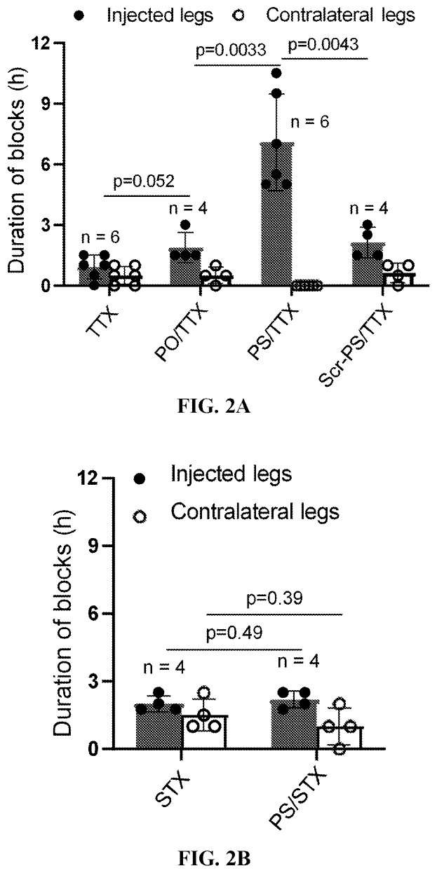

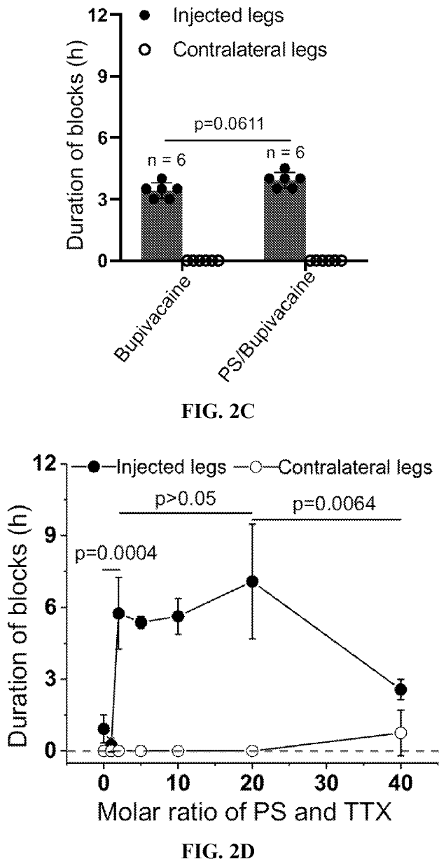

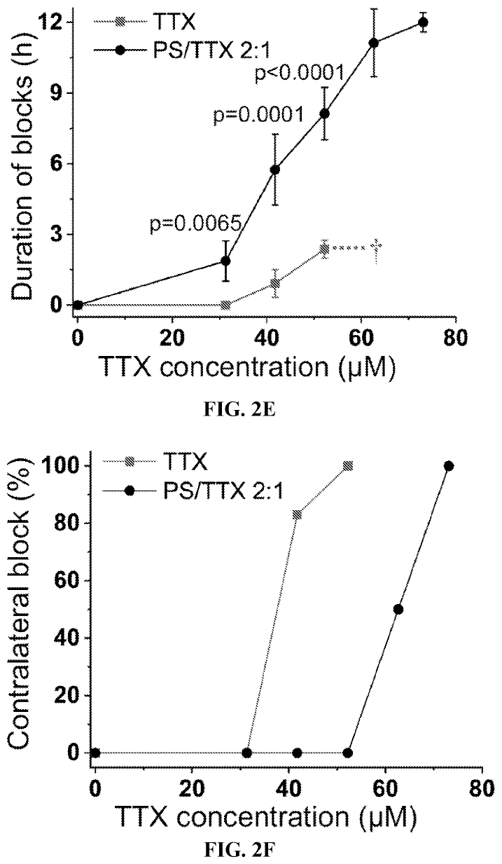

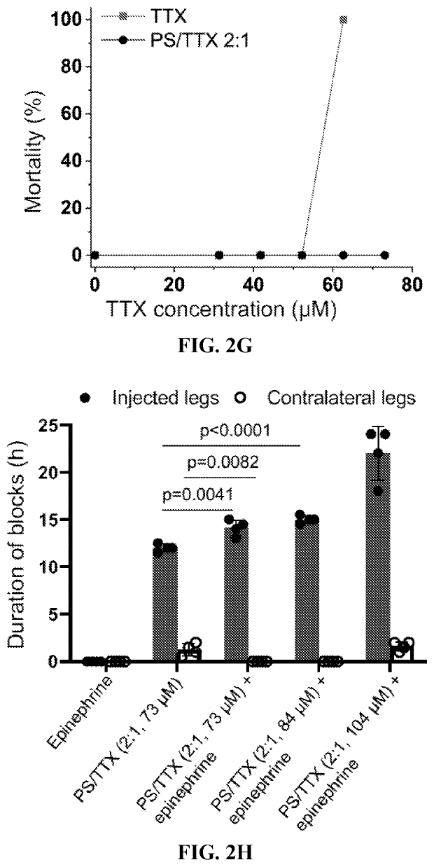

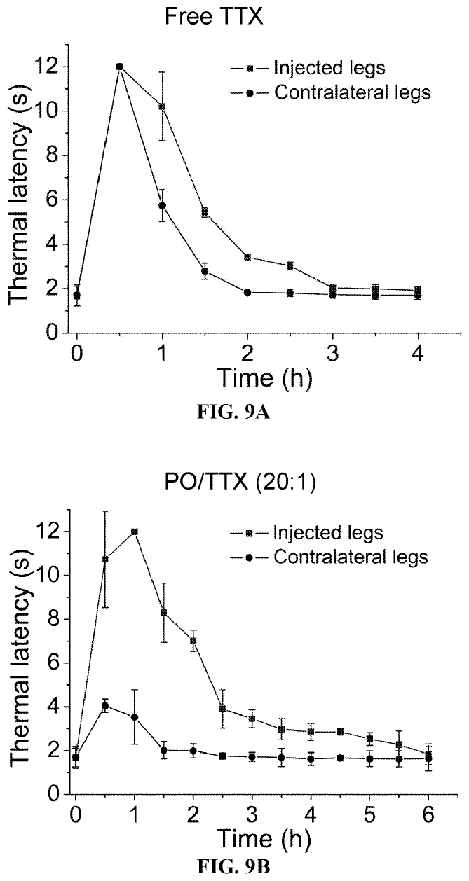

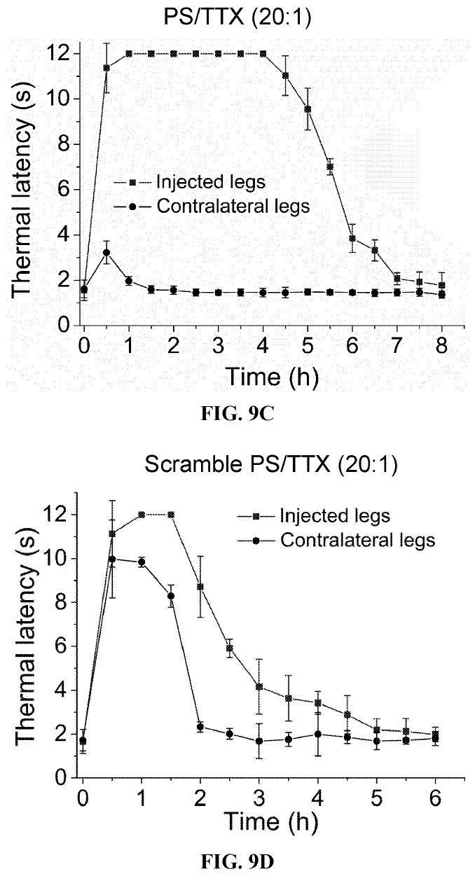

Nerve block with 42 μM free TTX lasted 0.9±0.6 h. Delivery as PO/TTX doubled block duration to 1.9±0.8 h, and as PS/TTX increased it 7.7-fold, to 7.1±2.4 h (FIG. 2A and FIGS. 9A-9D). Block from PS/TTX was statistically significantly longer than that from TTX (p=0.0001) or PO/TTX (p=0.0033).

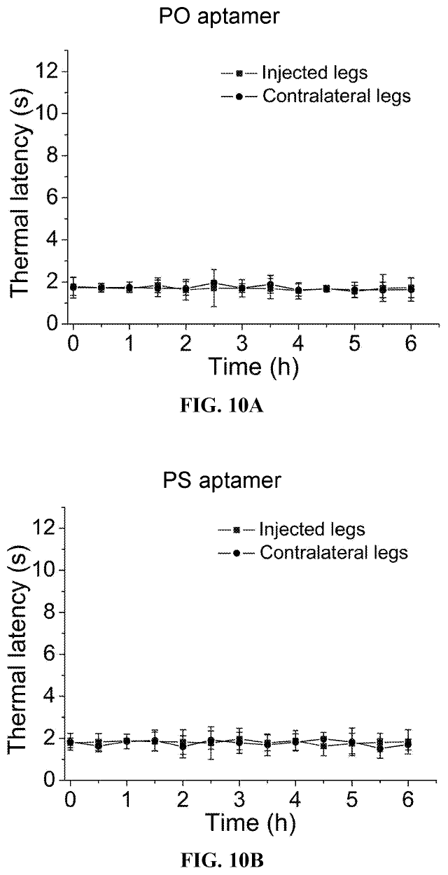

To assess the possibility that nerve blocks were resulting from an inherent aptamer property (rather than from TTX), rats were injected at the left sciatic nerve with PO or PS aptamers (83.6 μM or 836 μM) without TTX. No nerve block was detected over a 6 h period (FIGS. 10A-10B and Table 2).

### Example 3. Specificity of PS Aptamer/TTX In Vivo

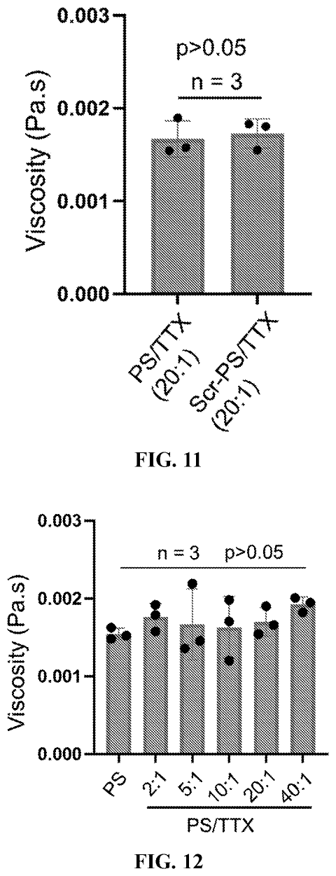

To determine the specificity of the TTX-binding PS aptamer, three more control groups were utilized to verify the selectivity of the exact aptamer to TTX in vivo. First, the non-selective scrambled PS aptamer mentioned above was complexed with TTX (Scr-PS/TTX) at a fixed molar ratio of 20:1. The Scr-PS/TTX (42 μM TTX) showed a 2.3-fold improvement of nerve block duration over free TTX (to 2.1±0.8 h, p=0.0207) which is likely due to non-specific interactions between Scr-PS and TTX, but lower than that of the more selective PS/TTX moiety (7.7-fold, 7.1±2.4 h, p=0.0043) (FIG. 2A and FIGS. 9A-9D). Of note, both PS/TTX (20:1) and Scr-PS/TTX (20:1) had a very similar and low viscosity (<0.002 Pa s) determined by rheometry (FIG. 11) which is not responsible for the differences of durations of nerve block. Second, the selectivity of the PS aptamer for TTX was confirmed by complexing it with another S1SCB, STX which acts at the same site on the sodium channel as TTX, has a similar molecular weight and guanidinium group but is otherwise structurally very different (FIG. 1C). 2 μg (22 μM) STX provided sensory nerve blockade lasting 2.0±0.4 h, while nerve block from the same concentration of STX in a 20:1 molar ratio with the TTX-specific-PS (PS/STX) was unchanged at 2.2±0.4 h (p=0.49), showing that the TTX-binding aptamer did not prolong nerve block from STX (FIG. 2B). Third, an amino-amide local anesthetic, bupivacaine, was combined with the TTX-binding aptamer. It binds to the same sodium channel as TTX but at a different site on the inner surface of the cell membrane, and is structurally very different (FIG. 1C). The combination of 836 μM PS aptamer with 15.4 mM bupivacaine hydrochloride did not significantly prolong the duration of nerve block (3.9±0.4 h with PS/bupivacaine versus 3.4±0.4 h from free bupivacaine; p=0.0611) (FIG. 2C). These results confirm specific interactions between TTX and the TTX-binding PS aptamer.

### Example 4. Ratio of PS Aptamer and TTX to Enhance Nerve Blockade

The effect of the PS/TTX ratio (1:1, 2:1, 5:1, 10:1, 20:1, and 40:1) was investigated on the duration of nerve block, at a constant TTX concentration of 42 μM. These formulations were injected at the sciatic nerve site. PS/TTX with a molar ratio of 1:1 induced sensory nerve block lasting 0.3±0.3 h, which was similar to the duration of block from the same concentration of free TTX (FIG. 2D). In the range of PS/TTX ratios from 2:1 to 20:1, block was markedly prolonged: 5.8±1.5 h (2:1), 5.4±0.3 h (5:1), 5.6±0.8 h (10:1), 7.1±2.4 h (20:1). There was no statistically significant difference in the observed injected or contralateral latencies over that range of molar ratios. However, when the PS/TTX ratio was increased to 40:1, the duration of nerve block was reduced to 2.6±0.4 h. Consequently, PS/TTX with the molar ratio 2:1, which provided long nerve block time at the lowest aptamer concentration, was selected for subsequent studies.

All PS/TTX complexes had a very low viscosity less than 0.003 Pa s in the range of angular frequencies tested (FIG. 12). There was no difference in the measured viscosity of the complexes at different ratios (n=3, p>0.05 for all comparisons), so differences in viscosity did not cause the differences in nerve block.

### Example 5. Dose Response with Aptamer/TTX

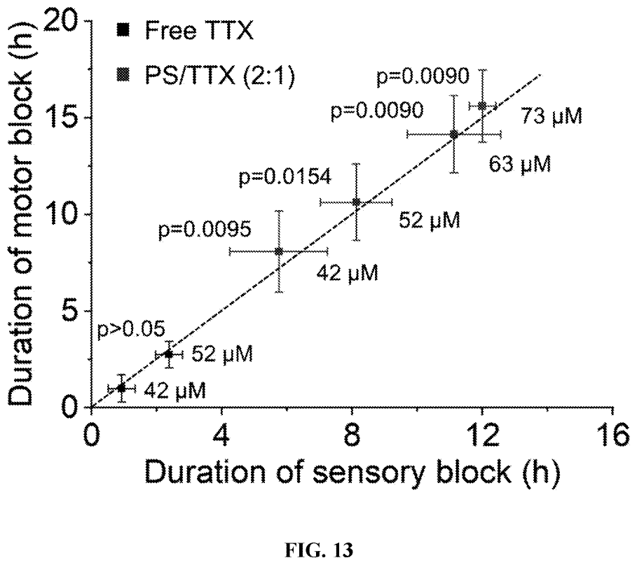

Dose-response experiments were performed in rat left sciatic nerve block model (n=4 or 6) with free TTX drug or PS/TTX (2:1) complexes. Nerve blocks on the injected side were much longer with PS/TTX than with free TTX at all dosages used (FIG. 2E and Table 2). The duration of nerve blockade increased with increasing PS/TTX dose (1.9±0.9 h at 31 μM TTX, 12.0±0.4 h at 73 μM TTX). PS enhanced the performance of low doses of TTX: 31 μM (3 μg) of free TTX caused no nerve block, while the same dose in PS/TTX produced a median duration of sensory nerve block of 1.9±0.9 h, with successful nerve block in 100% of animals. Additionally, PS/TTX improved the safety of TTX as evidenced by the absence or reduction of systemic toxicity from TTX (i.e., deficits in the contralateral extremities; FIG. 2F and Table 2). For example, 63 μM (6 μg) of free TTX was uniformly fatal, while no rats died at any dose of PS/TTX tested (FIG. 2G and Table 2). Contralateral latency was dose-dependent with both TTX and PS/TTX, but it was much greater with TTX than PS/TTX. Motor blocks were 1.3-fold longer than sensory blocks for PS/TTX-treated rats (p<0.05) (FIG. 13).

### Example 6. Potentiating Local Anesthetic Activity of PS/TTX by Addition of Epinephrine

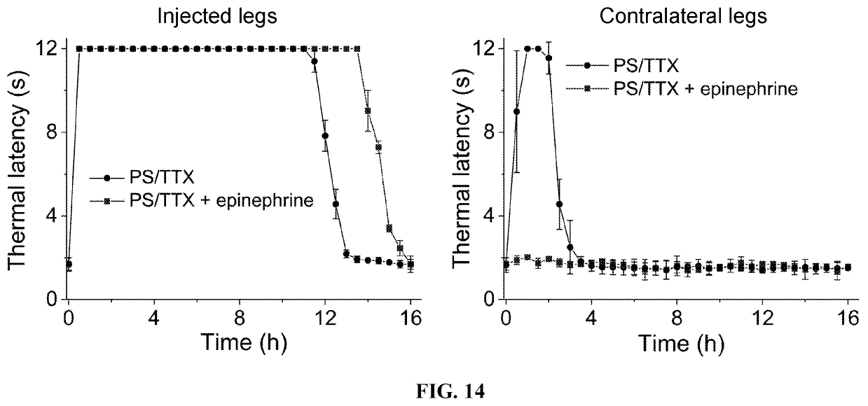

The prolongation of nerve block by PS is due to control of TTX release from the site of injection. This control allowed the delivery of doses of TTX that would otherwise be fatal. It was postulated that the dose delivered—and therefore the duration of effect—could be further increased by co-injection of an agent that could slow release from the site of injection by pharmacological means, epinephrine. Co-injection of PS/TTX (2:1) and 55 μM epinephrine at the sciatic nerve allowed TTX concentrations as high as 104 μM to be delivered without mortality (FIG. 2H). This is a dose where, in the absence of aptamer, TTX is uniformly fatal, and even with epinephrine administration there was a 67% fatality rate. A duration of block of 22.0±2.8 h could be achieved with 104 μM PS/TTX with epinephrine, almost twice the longest duration that could be achieved with PS/TTX without epinephrine and without animal mortality. Peak contralateral latency was markedly reduced by epinephrine, e.g., 12 s for 73 μM in PS/TTX and 2 s in PS/TTX with epinephrine (p<0.0001) (FIG. 14).

### Example 7. Tissue Retention of PS/TTX

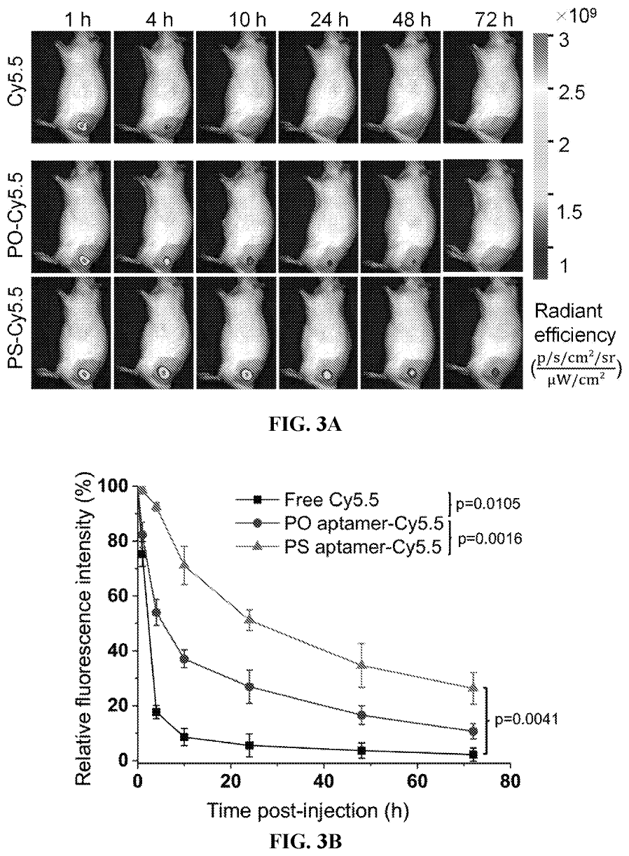

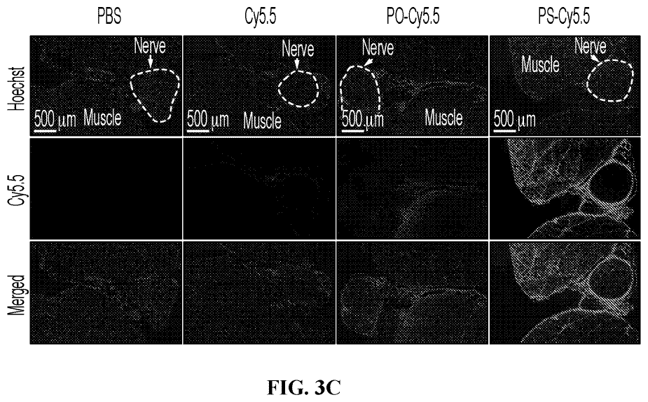

To assess the local retention of aptamers in tissue, rats were injected at the left sciatic nerve with PO and PS aptamers covalently modified with the near-infrared fluorescence dye Cy5.5 at their 5′ end (Cy5.5-PO/PS, Table 1). The fluorescence intensity at the injection site was monitored at predetermined intervals with an in vivo imaging system (IVIS). Free Cy5.5 was cleared rapidly with less than 20% left at 4 h, while approximately 63% of the PO aptamer was cleared within 10 h (FIG. 3A). With PS aptamer, ~50% of the peak fluorescence remained at 24 h. The fluorescence intensity of PS-Cy5.5 was statistically significantly stronger than for Cy5.5 and PO-Cy5.5 at all time-points tested (FIG. 3B). These data confirm that the PS aptamer allowed for longer tissue retention.

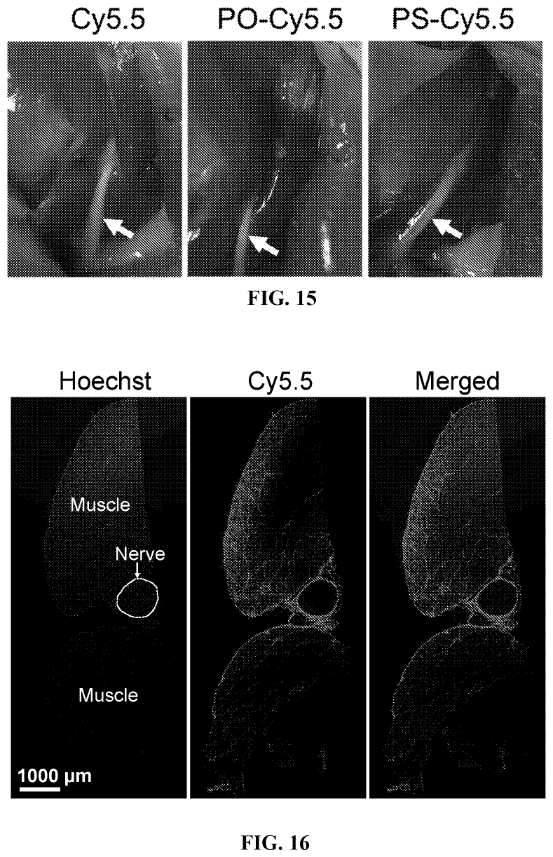

The sciatic nerves and surrounding tissues were harvested 4 h post-injection, processed for histology, stained with Hoechst 33342 (staining the nuclei), and imaged by laser scanning confocal microscopy. Fluorescence from Cy5.5 was detected in the tissues surrounding the sciatic nerve in PS aptamer-injected rats that was ~4-fold higher than that in animals injected with the PO aptamer (FIG. 3C and FIG. 15). Aptamer fluorescence was observed in the connective tissue between muscle and nerve (i.e., at the site of injection near the nerve), but not within the sciatic nerve itself (FIG. 3C and FIG. 16).

The presence of TTX in tissue at pharmacologically relevant concentrations is revealed by the neurobehavioral bioassay: while nerve block persists, TTX is present: when nerve block resolves, the TTX/STX is essentially gone.

### Example 8. Tissue Reaction

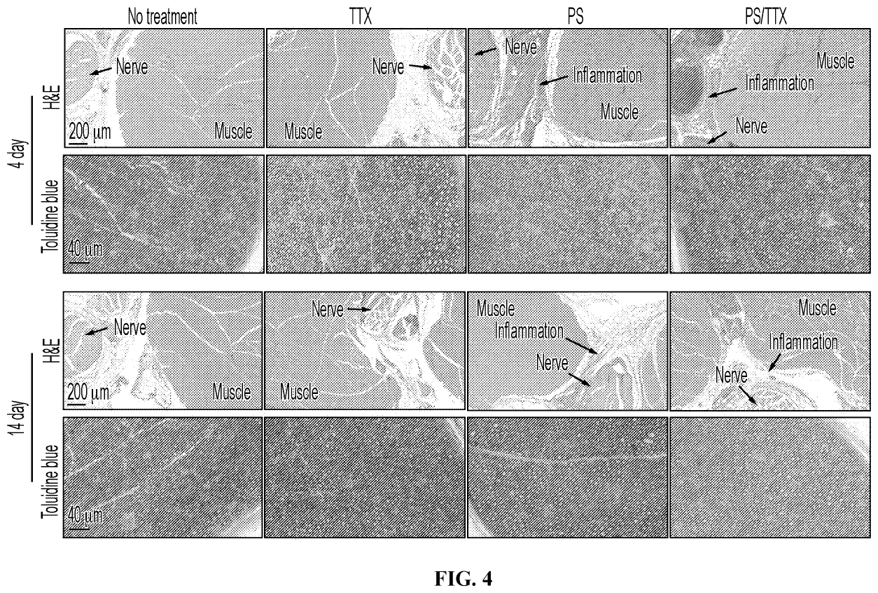

The sciatic nerve with surrounding tissues were harvested at days 4 and 14 after sciatic nerve injection and processed for histology (n=4 for all groups; FIG. 4; Table 3). Muscle tissue was stained with hematoxylin-eosin (H&E). Free TTX induced very mild inflammation (score=0 or 1 in all animals), which is consistent with previous reports. A mixed inflammatory response (score=2 or 3 in all animals) was observed at the site of injection for the PS aptamer and PS/TTX groups at day 4. Inflammation diminished (score=1 or 2 in all animals) by day 14 (n=4, p<0.05 compared to day 4). There was no myotoxicity in any treatment groups (score=0) or untreated rats at any time point (Table 3). To examine neurotoxicity, the sciatic nerves were embedded in Epon and stained with toluidine blue (H&E staining is relatively insensitive for identifying nerve injury.). The PS aptamer and PS/TTX complexes did not cause any nerve injury at either time point (FIG. 4).

### Example 9. In Vivo Nerve Block of STX-Binding PS Aptamer (PSAP_{STX})/STX Complexes

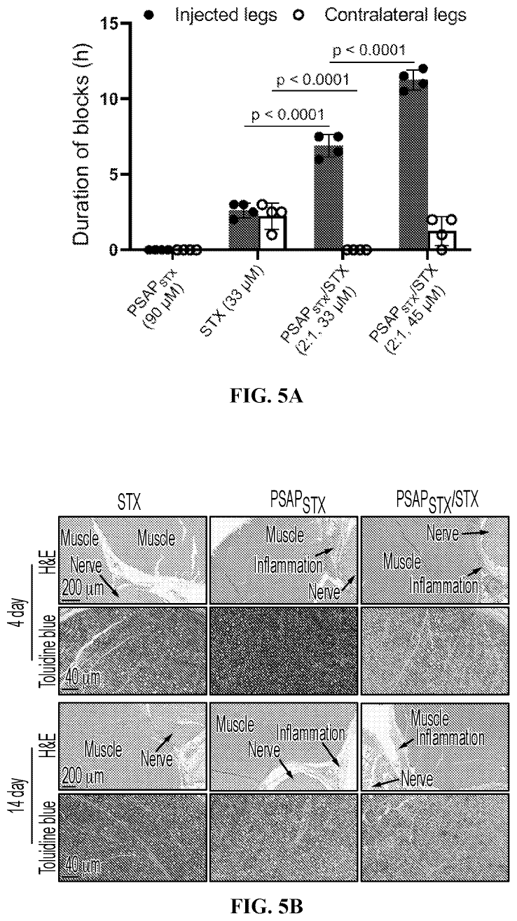

To test the generality of the use of aptamers as drug delivery systems, peripheral nerve block with STX was investigated with a PS aptamer specific to STX (PSAP_{STX}, Table 1). STX has approximately twice the potency of TTX in vivo for rat sciatic nerve block. Injection of 33 μM free STX in PBS caused nerve block lasting 2.6±0.5 h (FIG. 5A and Table 4), with contralateral deficits in all animals (with a duration of 2.3±0.9 h). In contrast, 33 μM STX in PSAP_{STX}/STX (molar ratio 2:1) increased nerve block in the injected extremity 2.6-fold, to 6.9±0.8 h (p<0.0001), and the animals had no increase in contralateral latency (FIG. 5A). PSAP_{STX}/STX (2:1) with 45 μM STX provided sensory nerve blockade lasting 11.3±0.7 h and while there were increases in contralateral latency, no animal died, whereas injection of free STX at the same dose was uniformly fatal.

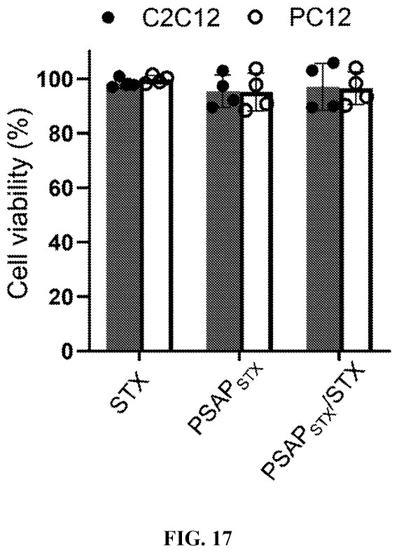

PSAP_{STX} and PSAP_{STX}/STX had no cytotoxicity in C2C12 and PC12 cell lines by MTS assay (FIG. 17). To assess tissue reaction, rats were euthanized 4 and 14 days after injection (n=4 at each time point). The sciatic nerve and surrounding tissues were harvested and processed for histology by H&E staining (for inflammation and myotoxicity) or toluidine blue staining (for neurotoxicity). Inflammation was similar to what was seen with PS/TTX. Similarly, there was no myotoxicity or neurotoxicity in any animals treated with free PSAP_{STX} or PSAP_{STX}/STX complexes at days 4 and 14 (FIG. 5B and Table 5).

### Example 10. Discussion of Examples 1-9

The use of aptamers to create highly effective depot-type drug delivery systems is demonstrated herein. Control of drug release was achieved by forming aptamer/drug non-covalent complexes, and in vivo results demonstrated that the efficacy and therapeutic index of small molecule drugs (TTX and STX) had been enhanced remarkably in a rat sciatic nerve block model.

This approach was effective with two molecules that are of a class that is difficult to encapsulate in many traditional drug delivery systems: small, hydrophilic molecules. Moreover, incorporation of such molecules in many traditional drug delivery systems may result in marked burst release, potentially causing toxicity. A number of approaches have been developed to improve their incorporation, relying on approaches such as electrostatic interactions with charged compounds, or covalent tethering to a polymer. These approaches vary considerably in the degree to which they are effective or complicated. The approach disclosed herein is straightforward and effective.

The aptamer DDS described here has several advantages over existing systems. Aptamers are easy to synthesize and chemically modify. These aptamers were synthesized through solid-phase technology and phosphoroamidite chemistry, which allows the addition of multiple functional moieties at designated positions. These synthetic techniques are simple, efficient, easy to scale-up and inexpensive. Importantly, the process of drug loading is simple one-step mixing, which has clear advantages over many others, which are often multi-step and have relatively low efficiencies.

The drug-aptamer interaction is the basis for the controlled release functionality of this system. Consequently, this approach could be used for a wide range of therapeutics. Here, local anesthesia was used as the in vivo model in which to demonstrate effect and safety. However, this technology could also be used for treatment of many other types of diseases, at least—based on these results—where depot-type drug delivery systems are desirable, whether to local effect (as in this case), or systemic. In systemic administration (i.e., intravenous injection of drug-aptamer complexes), the use of the aptamer's specific interaction to bind drug would likely mean that the aptamer could not be used to target specific tissues.

PS chemical modification improves the in vivo stability of aptamers against degradation by nucleases. PS modification can increase the binding affinity between aptamer and drug, improving drug release kinetics. It is believed that the enhanced affinity of the PS aptamer to TTX can be attributed to the changed electrostatic and hydrophobic interactions between them due to the oxygen-to-sulfur substitutions in the DNA backbone. This was seen with TTX (FIGS. 1D and 1E) and resulted in prolongation of effect and reduction of toxicity (FIG. 2E). PS aptamers also demonstrated much longer tissue retention than PO aptamers (FIG. 3). Two potential explanations are that the PS backbone confers aptamer resistance against nuclease digestion, and that PS has a high propensity to bind various proteins nonspecifically in vivo, which enhances tissue retention. It is not clear how much the enhanced tissue retention was beneficial in prolonging nerve block from TTX, since the timeframe of nerve block was much shorter than the time frame of tissue retention, but it could be important for drugs with longer durations of effect.

Tissue reaction to the PS aptamer was benign at the doses tested when used locally at the rat sciatic nerve. Moreover, there is a track-record of aptamers in clinical use. Therefore, safety issues are unlikely to be an obstacle in translation of an aptamer-based drug delivery system.

Thus, presented herein is the use of drug-specific DNA aptamers as drug delivery systems. This approach provides a simple and safe method of delivery for molecular payloads such as small molecule drugs.

### Example 11. Methods Used in Examples 1-10

### Materials

Phosphoramidites and supplies for aptamer synthesis were purchased from Glen Research Co., USA. Mouse C2C12 myoblast (CRL-1772) and rat PC12 pheochromocytoma (CRL-1721) cell lines were purchased from American Type Culture Collection (Rockville, MD, USA). Sulfo-Cyanine5.5 amine (Cy5.5, 95.0%) was acquired from Lumiprobe Corporation (Hunt Valley, Maryland, USA). Tetrodotoxin (TTX) was purchased from Abcam (Waltham, MA, USA). TTX ELISA kits were purchased from Reagen LLC (Moorestown, NJ, USA). Dulbecco's minimum essential medium (DMEM), horse bovine serum (HBS), fetal bovine serum (FBS), and Penicillin Streptomycin were purchased from Thermo Fisher Scientific Inc. (Waltham, MA, USA). STX was obtained from the U.S. Food and Drug Administration (FDA). Fluorescein sodium salt and phosphate buffered saline (PBS) were purchased from Sigma-Aldrich Co. (MO, USA). MALDI-TOF MS measurements were performed on a Bruker Microflex LT mass spectrometer (Bruker Daltonics Inc., MA, USA). Reverse-phase HPLC was carried out at a Waters (Waters Co., MA, USA) Breeze 2 HPLC system coupled to a SunFire C18 5 μm, 10×100 mm reverse phase column and a 2998 PDA detector, using TEAA buffer (0.1 M) and HPLC-grade acetonitrile as mobile phases. DLS data were recorded on Malvern Zetasizer Pro. Histology studies were carried out at the Kock Institute Swanson Biotechnology Center of Massachusetts Institute of Technology and at iHisto Inc.

### Aptamer Synthesis

The synthesis of DNA aptamers (both phosphodiester [PO] and phosphorothioate [PS] versions) was performed on a Model 391 DNA synthesizer (Applied Biosystems, Inc., CA, USA) using standard solid phase phosphoramidite methodology. Deprotection of aptamers was carried out with ammonium hydroxide (28% NH₃ in H₂O) for 24 h at room temperature. The crude products were purified by reverse-phase HPLC liquid chromatography. Subsequently, the purified aptamers were treated with 20% acetic acid in H₂O for 1 h to remove the dimethoxytrityl (DMT) protecting group, followed by extraction with ethyl acetate three times in an aqueous solution. The resultant aptamers were quantified using NanoDrop™ OneC microvolume UV-vis spectrophotometer and stored at −20° C. after lyophilization. To synthesize the dye-labeled aptamer, cyanine 5 (Cy5) or cyanine 5.5 (Cy5.5) was incorporated at the 5′-terminus by using Cy 5 phosphoramidites and Cy 5.5 phosphoramidites, respectively. The successful syntheses of all aptamers in this work were confirmed by MALDI-TOF MS.

### Preparation of Aptamer/TTX and Aptamer/STX Complexes

Briefly, the TTX-binding aptamers (PO or PS) were dissolved in 1×PBS (154 mM NaCl, 5.6 mM Na₂HPO₄, 1.1 mM KH₂PO₄; pH 7.4) in microcentrifuge tubes and heated to 95° C. for 5 min, followed by slow cooling to room temperature. After the annealing process, the TTX, dissolved in citrate buffer (5 mg/mL), was diluted with PBS (200 μg/mL) and then added to the aptamer solution at a predetermined molar ratio. The resulting solution was gently shaken overnight at room temperature for further tests. Similarly, the aptamer/STX complexes were prepared using the identical method.

### Microscale Thermophoresis (MST) Analysis of Binding Interactions

The immobilization-free MST is a rapid and precise method to study the small molecule-aptamer interactions in solution. It monitors the thermophoretic movement of different molecular ratios between target molecule and ligand through m-sized temperature gradients. To establish these ratios, a constant amount of Cy5-labeled aptamer is mixed with different amounts of ligand. A serial dilution of TTX in the PBS was carried out to provide solutions having a range of concentrations between 152.6 nM and 5 mM. 5 μl of each solution was mixed with 5 μl of the Cy5-labeled aptamer, which was held at a constant concentration of 16 nM. The final concentrations of TTX in each capillary ranged from 76.3 nM to 2.5 mM. Each sample was analyzed on a Monolith NT. Automated (NanoTemper Technologies, Munich, Germany) at 25° C., with 40% LED power and 80% laser power. Data were fitted using MO. Affinity Analysis software (version 2.3, NanoTemper Technologies) and MST-on time was set at 1.5 s to determine the aptamer K_{d} values.

### In Vitro TTX Release

The TTX release kinetics from aptamer/TTX complexes was determined by placing 200 μL of these complexes (TTX, 42 μM) into a Slide-A-Lyzer MINI dialysis device with a 3500 MW cut-off, further dialyzed with 14 mL PBS and incubated at 37° C. on a platform shaker. At predetermined intervals, the dialysis solution was exchanged with fresh, pre-warmed PBS. The concentration of released TTX in media was quantified by an enzyme-linked immunosorbent assay (ELISA).

### Determination of Viscosity

The rheological properties of aptamers and aptamer/TTX were measured using an AR2000 rheometer (TA Instruments, New Castle, DE, USA) with parallel plate geometry and a temperature controller. A parallel plate with a diameter of 20 mm was used, between which the gap distance was set as 0.3 mm. The dynamic properties were followed as a function of time at a constant shear stress of 0.1 Pa and an oscillation frequency of 0.01 rads⁻¹, in frequency sweep tests (in the frequency ranging from 0.01 to 100 rads⁻¹) at room temperature.

### Cell Culture

C2C12 mouse myoblasts were cultured in Dulbecco's modified Eagle's medium (DMEM) supplemented with 20% fetal bovine serum (FBS) and 1% Penicillin Streptomycin. To induce differentiation into myotubes, C2C12 cells (8.0×10³) were seeded into 24-well plates and incubated in DMEM with 2% horse serum and 1% Penicillin Streptomycin for 7-10 days. The differentiation media was exchanged every 2 to 3 days. PC12 rat adrenal gland pheochromocytoma cells were grown in DMEM with 5% FBS, 5% horse serum, and 1% Penicillin Streptomycin. For neuronal induction, PC12 cells were seeded in 24-well plates at a density of 1.0×10⁴ cells per well and cultured in DMEM with 5% FBS, 5% horse serum, and 50 ng/mL nerve growth factor (NGF) for 10-14 days.

### MTS Cytotoxicity Assay

The cytotoxicity of aptamers, TTX, and aptamer/TTX complexes was evaluated with the MTS colorimetric assay. The C2C12 and PC12 cells were treated with varying doses of free TTX, aptamers, or the aptamer/TTX complexes. Cells incubated with a vehicle (PBS) were set as a control. After 24 h of incubation, 40 μL of the MTS-based CellTiter 96® AQᵤₑₒᵤₛ One Solution Reagent was added to each well. The cells were incubated for another 4 h, and the absorbances (490 nm) were measured on a BioTek® Synergy™ Mx microplate reader (BioTek Inc., VT, USA).

### Animal Studies

Animal studies were approved by the Boston Children's Hospital Animal Care and Use Committee and carried out following protocols in accordance with the guidelines of the International Association for the Study of Pain. Adult male Sprague-Dawley rats (Charles River Laboratories, Wilmington, MA, USA) weighing 400-500 g were housed in groups under a 12-h/12-h light/dark cycle with lights on at 6:00 AM.

### Sciatic Nerve Block and Neurobehavioral Testing

The rats were randomly assigned to each group and injected with 300 μL of each formulation at the left sciatic nerve under brief isoflurane-oxygen anesthesia. A 23 G needle was introduced postero-medial to the greater trochanter, pointing in the anteromedial direction. Upon touching the bone, the drugs were injected onto the sciatic nerve. Then the rats underwent neurobehavioral testing at predetermined intervals.

The sensory nerve block was assessed using a modified hotplate test as described previously. Briefly, the plantar surface of the rat's hind paw was placed on a preheated hot plate (Model 39D Hot Plate Analgesia Meter; IITC) at 56° C. The time was recorded with a stopwatch when the rat withdrew its foot (the thermal latency). The paw was removed from the hot plate to avoid injury if the rat did not retract the foot after 12 s. This test was repeated three times (with a 10 s pause between tests) at each time interval. A thermal latency above 7 s indicated a successful nerve blockade for the purpose of calculating the duration of the nerve block.

The motor nerve block was evaluated using a weight-bearing test in which the motor strength of the rat's hindpaw was determined, as reported previously. Briefly, the rat was positioned with one hindpaw on a digital balance and was allowed to bear its own weight. The maximum weight that it could bear without the ankle touching the balance was recorded. The duration of motor blockade was defined as the time for weight-bearing to return halfway to normal from the maximal block as described previously.

### In Vivo Imaging System (IVIS) Imaging

The Sprague-Dawley rats were partly shaved and injected with 0.3 mL of free Cy5.5 or Cy5.5-labeled aptamers (Cy5.5, 4.64 μM) at the left sciatic nerve under the isoflurane-oxygen anesthesia. They were scanned at 0 h, 1 h, 4 h, 10 h, 24 h, 48 h, and 72 h post-injection using an IVIS 200 imaging system (Caliper Life Sciences, Inc. MA, USA). Quantitative analysis will be performed using the Live Imaging software of the IVIS. The half-life of tissue retention is the time required for the fluorescence intensity to decrease by 50% after injection and was calculated based on the fluorescence intensity.

### Tracking the Location of Aptamers in Tissue Using Confocal Imaging

Rats were injected with Cy5.5 or Cy5.5-labelled aptamers (84 μM) in 0.3 mL PBS at the left sciatic nerve under the isoflurane-oxygen anesthesia. They were then euthanized with carbon dioxide at 4 h post-injection. Sciatic nerves and surrounding tissues were harvested, embedded into the OCT compound (Fisher Scientific Inc., USA) and stored at −20° C. The frozen tissues were cut into 8 μm-thick sections using a cryostat microtome, which were mounted onto glass slides. The slides were then fixed with 4% paraformaldehyde, stained with Hoechst33342, and imaged on an LSM-880 confocal laser scanning microscopy (Carl Zeiss Ltd., Cambridge, UK).

### Histology

The Sprague-Dawley rats (n=4) were treated with 0.3 mL of aptamers (146 μM), free TTX (52 μM), or aptamer/TTX (146 μM/73 μM) at the left sciatic nerve, and then euthanized to assess the acute (at day 4) and chronic (at day 14) inflammation and tissue injury respectively. The sciatic nerve and surrounding tissue were collected, fixed in 10% neutral buffered formalin, and underwent standard processing for H&E-stained slide production. The slides were analyzed and scored for the presence of inflammation (0-4) and myotoxicity (0-6) by an independent, board-certified pathologist (Matthew Gregory Torre) who was blinded to the nature of the individual samples. The inflammation score is a subjective quantification of severity in which 0 was normal and 4 was severe inflammation (0: no inflammation, 1: peripheral inflammation, 2: deep inflammation, 3: muscular hemifascicular inflammation, 4: muscular holofascicular inflammation). The myotoxicity score is determined by the nuclear internalization and regeneration of myocytes. Nuclear internalization is characterized by myocytes having nuclei located away from their usual location at the periphery of the cell. Regeneration is characterized by the presence of shrunken myocytes with basophilic cytoplasm. The scoring scale is as follows: 0=normal; 1=perifascicular internalization; 2=deep internalization (more than five cell layers); 3=perifascicular regeneration; 4=deep tissue regeneration (more than five cell layers); 5=hemifascicular regeneration; 6=holofascicular regeneration.

To access the neurotoxicity of the aptamers' formulations, the sciatic nerves were fixed in Karnovsky's K11 solution (1.25% formaldehyde, 2.5% glutaraldehyde, and 0.03% picric acid in 0.1 M sodium cacodylate buffer, pH 7.4.). The fixed tissues were washed with 0.1M sodium cacodylate buffer and post-fixed with 1% osmium tetroxide/1.5% potassium ferrocyanide (in H₂O) for 2 h. Samples were then washed in a maleate buffer and post-fixed in 1% uranyl acetate in maleate buffer for 1 h. Tissues were then rinsed in ddH₂O and dehydrated through a series of ethanol (50%, 70%, 95%, (2×)100%) for 15 minutes per solution. Dehydrated tissues were put in propylene oxide for 5 minutes before they were infiltrated in epon mixed 1:1 with propylene oxide overnight at 4° C. Samples were polymerized in a 60° C. oven in epon resin for 48 h. They were then sectioned into 500 nm thin sections, which were stained with toluidine blue and imaged on high-resolution light microscopy.

### Statistics and Reproducibility

All quantitative measurements (i.e., TTX binding affinity analysis, TTX release, quantification of the fluorescence intensity) have at least three independent repeats. Sample size in Examples 1-10 was calculated using a power analysis. Previous animal studies, or small pilot studies, when necessary, served as the basis for calculations of expected averages and deviations used to calculate power, for which experiment studies were set to a value of 0.8. This typically resulted in an experimental group size to be n=4-6, depending on the experiment. Sample size is explicitly stated for each experimental group for individual experiments in the Brief Descriptions of the Drawings and Examples 1-10. No data were excluded from the analyses. Origin 2022b and GraphPad Prism 9 were used for plotting. ImageJ (Version 1.53t) was used for image processing. MO. Affinity Analysis (version 2.3) software was used for binding affinity analysis. Statistical comparisons were performed using GraphPad Prism 9. Statistical comparisons were performed using Student t-test (two-sided) unless stated otherwise. Thermal latency, inflammation, and myotoxicity scores are reported as medians and quartiles due to their ordinal or non-Gaussian character. Data are presented as mean±standard deviation. Statistical significance was set at p<0.05.

### Example 12. Tables Referenced in Examples 1-11

TABLE 1
DNA aptamers sequences

| Strand | |
|---|---|
| description | Sequence |
| TTX-binding | 5′-AAAAATTTCACACGGGTGCCTCGGCTGTCC-3′ (SEQ ID NO: 1) |
| PO aptamer | |
| Cy5.5-labeled | 5′-Cy5.5-AAAAATTTCACACGGGTGCCTCGGCTGTCC-3′ (SEQ ID NO: 1) |
| TTX-binding | |
| PO aptamer | |
| Cy5-labeled | 5′-Cy5-AAAAATTTCACACGGGTGCCTCGGCTGTCC-3′ (SEQ ID NO: 1) |
| TTX-binding | |
| PO aptamer | |
| TTX-binding | 5′-A*A*A*A*A*T*T*T*C*A*C*A*C*G*G*G*T*G*C*C*T*C*G*G*C*T*G*T*C*C-3′ |
| PS aptamer | (SEQ ID NO: 2) |
| Cy5.5-labeled | 5′-Cy5.5-A*A*A*A*A*T*T*T*C*A*C*A*C*G*G*G*T*G*C*C*T*C*G*G*C*T*G*T*C*C-3′ |
| TTX-binding | (SEQ ID NO: 2) |
| PS aptamer | |
| Cy5-labeled | 5′-Cy5-A*A*A*A*A*T*T*T*C*A*C*A*C*G*G*G*T*G*C*C*T*C*G*G*C*T*G*T*C*C-3′ |
| TTX-binding | (SEQ ID NO: 2) |
| PS aptamer | |
| Scrambled PS | 5′-G*G*C*T*C*C*A*C*T*C*A*T*A*C*T*T*C*C*G*A*G*T*A*A*G*A*C*G*G*T*-3′ |
| aptamer | (SEQ ID NO: 3) |
| Cy5-labeled | 5′-Cy5-G*G*C*T*C*C*A*C*T*C*A*T*A*C*T*T*C*C*G*A*G*T*A*A*G*A*C*G*G*T*-3′ |
| scrambled PS | (SEQ ID NO: 3) |
| aptamer | |
| Complementary | 5′-G*G*A*C*A*G*C*C*G*A*G*G*C*A*C*C*C*G*T*G*T*G*A*A*A*T*T*T*T*T*-3′ |
| strand of TTX- | (SEQ ID NO: 4) |
| binding PS | |
| aptamer | |
| STX-binding | 5′-T*T*G*A*G*G*G*T*C*G*C*A*T*C*C*C*G*T*G*G*A*A*A*C*A*G*G*T*T*C*A*T*T*G-3′ |
| PS aptamer | (SEQ ID NO: 5) |

Asterisk (*): phosphorothioate internucleotide linkage; PO: phosphodiester; PS: phosphorothioate; Scrambled PS aptamer: randomly rearranging the nucleotide sequence of TTX-binding PS aptamer

TABLE 2
Efficacy of TTX or aptamer/TTX conjugates in sciatic nerve blockade.

| | | Sensory | % Blocks | % | | |
|---|---|---|---|---|---|---|
| | TTX | Block | injected | Contralateral | | Mortality |
| Sample | (μM) | time (h) | legs | block | N | (%) |
| Free TTX | 31 | 0 | 0 | 0 | 4 | 0 |
| | 42 | 0.9 ± 0.6 | 83 | 83 | 6 | 0 |
| | 52 | 2.4 ± 0.4 | 100 | 100 | 6 | 0 |
| | 63 | — | — | — | 4 | 100 |
| PS/TTX 1:1 | 42 | 0.3 ± 0.3 | 50 | 0 | 4 | 0 |
| PS/TTX 2:1 | 31 | 1.9 ± 0.9 | 100 | 0 | 4 | 0 |
| | 42 | 5.8 ± 1.5 | 100 | 0 | 4 | 0 |
| | 52 | 8.1 ± 1.1 | 100 | 0 | 4 | 0 |
| | 63 | 11.1 ± 1.4 | 100 | 50 | 4 | 0 |
| | 73 | 12 ± 0.4 | 100 | 100 | 4 | 0 |
| PS/TTX 5:1 | 42 | 5.4 ± 0.3 | 100 | 0 | 4 | 0 |
| PS/TTX 10:1 | 42 | 5.6 ± 0.8 | 100 | 0 | 4 | 0 |
| PS/TTX 20:1 | 42 | 7.1 ± 2.4 | 100 | 0 | 6 | 0 |
| PO/TTX 20:1 | 42 | 1.9 ± 0.8 | 100 | 75 | 4 | 0 |
| Scr-PS/TTX 20:1 | 42 | 2.1 ± 0.8 | 100 | 75 | 4 | 0 |
| PS/TTX 40:1 | 42 | 2.6 ± 0.4 | 100 | 0 | 4 | 0 |
| PO aptamer | 0 | 0 | 0 | 0 | 4 | 0 |
| PS | 0 | 0 | 0 | 0 | 4 | 0 |
| aptamer | | | | | | |

TABLE 3
Myotoxicity and inflammation for TTX formulations.

| | | | Myotoxicity | Myotoxicity | Inflammation | Inflammation |
|---|---|---|---|---|---|---|
| | TTX | Aptamer | score | score | score | score |
| Sample | (μM) | (μM) | Day 4 | Day 14 | Day 4 | Day 14 |
| No | 0 | 0 | 0 (0-0) | 0 (0-0) | 0 (0-0) | 0 (0-0) |
| treatment | | | | | | |
| Free TTX | 52 | 0 | 0 (0-0) | 0 (0-0) | 0.5 (0-1) | 0 (0-0) |
| P value | | | >0.05 | >0.05 | >0.05 | >0.05 |
| TTX- | 73 | 146 | 0 (0-0) | 0 (0-0) | 2.5 (2-3) | 1 (1-2) |
| binding PS | | | | | | |
| aptamer | | | | | | |
| P value | | | >0.05 | >0.05 | <0.05 | <0.05 |
| PS/TTX | 73 | 146 | 0 (0-0) | 0 (0-0) | 3 (2-3) | 1 (1-2) |
| P value | | | >0.05 | >0.05 | <0.05 | <0.05 |

Inflammation scores: 0-4; myotoxicity scores: 0-6.
Data are medians with 25^{th} and 75^{th} percentiles in parentheses.
P values are for the comparison of the tissue reaction of test compounds to that of the untreated group; n = 4 rats for all groups.
Statistical comparisons were performed using Student t-test (two-sided).

TABLE 4
Nerve block in injected and contralateral hindpaws after
injection of STX or STX-binding aptamer/STX conjugates.

| | | Sensory | % Blocks | % | | |
|---|---|---|---|---|---|---|
| | STX | Block time | injected | Contralateral | | Mortality |
| Sample | (μM) | (h) | legs | block | N | (%) |
| STX | 33 | 2.6 ± 0.5 | 100 | 100 | 4 | 0 |
| | 45 | — | — | — | 4 | 100 |
| PSAP_{STX}/STX | 33 | 6.9 ± 0.8 | 100 | 0 | 4 | 0 |
| (2:1) | 45 | 11.3 ± 0.6 | 100 | 75 | 4 | 0 |
| PSAP_{STX} | 90 | 0 | 0 | 0 | 4 | 0 |

Data are means ± s.d.
PSAP_{STX}: PS aptamer specific to STX.

TABLE 5
Myotoxicity and inflammation for STX formulations.

| | | | Myotoxicity | Myotoxicity | Inflammation | Inflammation |
|---|---|---|---|---|---|---|
| | TTX | Aptamer | score | score | score | score |
| Sample | (μM) | (μM) | Day 4 | Day 14 | Day 4 | Day 14 |
| No treatment | 0 | 0 | 0 (0-0) | 0 (0-0) | 0 (0-0) | 0 (0-0) |
| STX | 33 | 0 | 0 (0-0) | 0 (0-0) | 0.5 (0-1) | 0 (0-0) |
| P value* | | | >0.05 | >0.05 | >0.05 | >0.05 |
| PSAP_{STX} | 0 | 90 | 0 (0-0) | 0 (0-0) | 2.5 (2-3) | 2 (2-2) |
| P value* | | | >0.05 | >0.05 | <0.05 | <0.05 |
| PSAP_{STX}/ | 45 | 90 | 0 (0-0) | 0 (0-0) | 2 (2-3) | 1 (1-2) |
| STX | | | | | | |
| P value* | | | >0.05 | >0.05 | <0.05 | <0.05 |

Inflammation scores: 0-4; myotoxicity scores: 0-6.
Data are medians with 25^{th} and 75^{th} percentiles in parentheses.
*P values are for the comparison of the tissue reaction of test compounds to that of the untreated group; n = 4 rats for all groups.
Statistical comparisons were performed using Student t-test (two-sided).

### Additional Aptamer-Drug Complexes

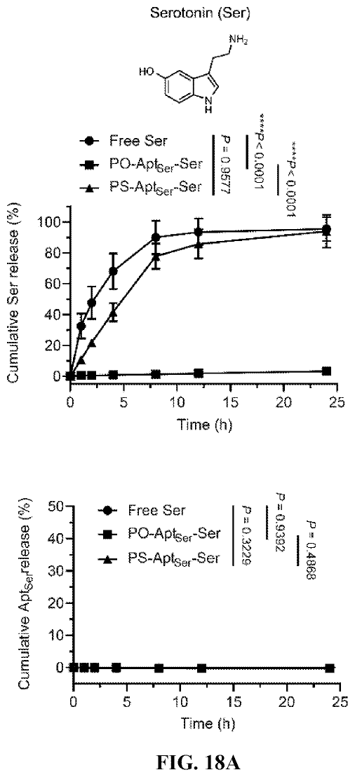

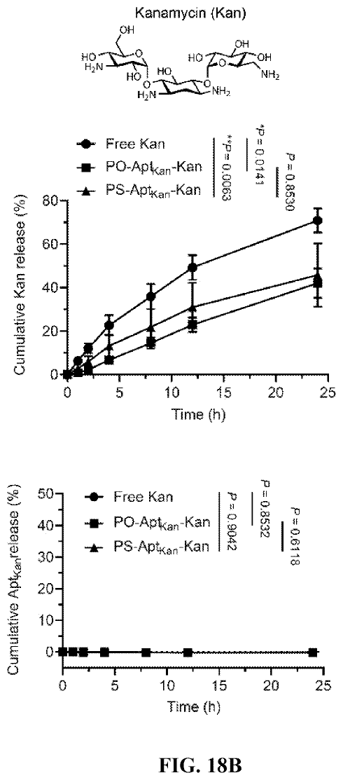

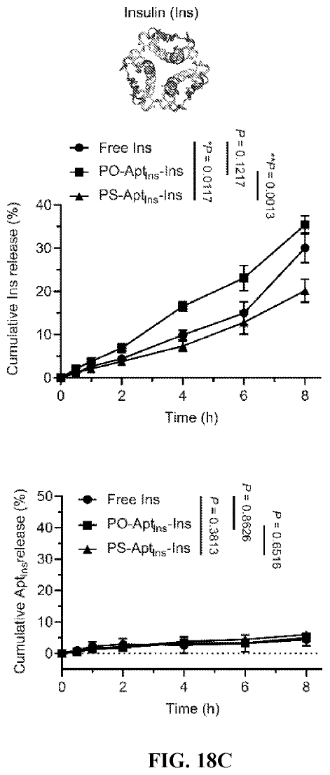

The feasibility of aptamers for sustained release of various type of therapeutics beyond local anesthetics was investigated. To this end, aptamers known to bind the neurotransmitter serotonin (Ser), the antibiotic kanamycin (Kan), and the anti-diabetic drug insulin (Ins) were selected (Table 6). The sustained release of a small molecule drug, Ser, from the aptamer system was first investigated. The Ser-binding aptamer (Apt_{Ser}) was mixed with Ser at a molar ratio of 2:1 and incubated at 4° C. overnight. The release kinetics of the resulting Apt-Ser complexes were evaluated by dialyzing 250 μL of them against 25 mL of PBS. PS-Apt_{Ser}-Ser showed slower Ser release than the free Ser initially but became similar at 24 h (P=0.9577. FIG. 18A). In contrast, PO-Apt_{Ser}-Ser showed dramatically slower Ser release than free Ser, with a cumulative Ser release as low as 3.31±1.61% at 24 h. The insufficient sustained release of PS-Apt_{Ser}-Ser could be attributed to PS-modification-induced disruption of the tertiary conformation of the aptamer in this specific sequence. The sustained release of another small molecule drug, Kan, from the Kan-binding aptamer (Apt_{Kan}) system using a similar experimental setup was then investigated. While PS-Apt_{Kan}-Kan and PO-Apt_{Kan}-Kan displayed similar Kan release at 24 h (P=0.8530, FIG. 18B), they both had slower release compared to free Kan (P=0.0141 and P=0.0.063).

Having demonstrated that the aptamers could act as sustained release system for the small molecule drug Ser and Kan, the feasibility of Ins-binding aptamer (Apt_{Ins})-mediated sustained release of protein drug Ins using a similar experimental setup was further investigated. The release of Ins from PS-Apt_{Ins}-Ins was significantly less than that from the free Ins at 24 h (P=0.0117, FIG. 18C). Meanwhile, the release of Ins from PO-Apt_{Ins}-Ins and free Ins was not significantly different at 24 h (P=0.1217). Compared to conventional release systems, the aptamer release system was relatively small, with a molecular weight of about 10 kDa. Thus, it is necessary to ensure that aptamers, as the drug loading and release platforms, were not released from the dialysis device. Indeed, the release of aptamers from the dialysis device was not observed for all the aptamer systems (FIG. 18A-C). Accordingly, the controlled release of all three drugs were achieved by the aptamers.

TABLE 6

| Sample | Sequence (5′-3′) |
|---|---|
| Serotonin-binding | CGACTGGTAGGCAGATAGGGGAAGCTGATTCGATGCGTGGGTCG |
| PO-Apt (PO- | (SEQ ID NO: 6) |
| AptSer) | |
| Serotonin-binding | C*G*A*C*T*G*G*T*A*G*G*C*A*G*A*T*A*G*G*G*G*A*A*G*C*T*G*A*T*T* |
| PS-Apt (PS-AptSer) | C*G*A*T*G*C*G*T*G*G*G*T*C*G (SEQ ID NO: 7) |
| Kanamycin- | TGGGGGTTGAGGCTAAGCCGA (SEQ ID NO: 8) |
| binding PO-Apt | |
| (PO-AptKan) | |
| Kanamycin- | T*G*G*G*G*G*T*T*G*A*G*G*C*T*A*A*G*C*C*G*A (SEQ ID NO: 9) |
| binding PS-Apt | |
| (PS-AptKan) | |
| Insulin-binding | GGTGGTGGGGGGGGTTGGTAGGGTGTCTTC (SEQ ID NO: 10) |
| PO-Apt (PO- | |
| AptIns) | |
| Insulin-binding PS- | G*G*T*G*G*T*G*G*G*G*G*G*G*G*T*T*G*G*T*A*G*G*G*T*G*T*C*T*T*C |
| Apt (PS-AptIns) | (SEQ ID NO: 11) |

Asterisk (*): phosphorothioate internucleotide linkage; PO: phosphodiester (original DNA backbone); PS: phosphorothioate (modified DNA backbone);

Serotonin ELISA kit was obtained from ImmuSmol (BA-E-5900R, France). Kanamycin ELISA kit (502370) was obtained from Cayman Chemical (Ann Arbor, MI). Insulin ELISA Kit was obtained from Cystal Chem (90095, Downers Grove). Quant-iT OliGreen ssDNA Assay Kit (O11492), insulin (RP-10935), were acquired from Thermo Fisher Scientific Inc. (Waltham, MA).

The release kinetics of serotonin, kanamycin, and insulin from aptamer/drug complexes (molar ratio, 2:1) were evaluated by placing 250 μL of these complexes (serotonin 94 μM; kanamycin 69 μM; insulin 7 μM) into a dialysis device (3.5-5 kDa cut-off for serotonin and kanamycin, 25 kDa cut-off for insulin). The dialysis device was then dialyzed against 25 mL PBS at 37° C. on a shaker. At predetermined intervals, the dialysis solution was collected, and fresh PBS was added to the system. The concentration of serotonin, kanamycin and insulin in the released media was quantified by the corresponding ELISA kits. The concentration of aptamers in the released media was quantified by a Quant-iT OliGreen ssDNA Assay kit.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A composition comprising an aptamer and a molecular payload, wherein the molecular payload is bound to the aptamer, and wherein the aptamer is a sustained release carrier for the molecular payload.

2. The composition of claim 1, wherein the aptamer is an oligonucleotide.

3. The composition of claim 1 or 2, wherein the aptamer is a single stranded oligonucleotide.

4. The composition of any of claims 1-3, wherein the aptamer is a deoxyribonucleic acid (DNA) or a ribonucleic acid (RNA).

5. The composition of any of claims 1-4, wherein the aptamer is a single-stranded deoxyribonucleic acid (DNA)

6. The composition of any of claims 1-5, wherein the aptamer is an oligonucleotide sequence of 15-60 nucleotides.

7. The composition of any of claims 1-6, wherein the aptamer is an oligonucleotide sequence of 30-35 nucleotides.

8. The composition of any of claims 1-7, wherein the aptamer is an oligonucleotide sequence having at least one phosphorothioate internucleotide linkage.

9. The composition of any of claims 1-8, wherein the aptamer is an oligonucleotide sequence having a complete phosphorothioate backbone.

10. The composition of any of claims 1-9, wherein the aptamer is a single-stranded deoxyribonucleic acid (DNA) having a complete phosphorothioate backbone.

11. The composition of any of claims 1-10 wherein the aptamer is SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

12. The composition of any of claims 1-11, wherein the molar ratio of aptamer to molecular payload is greater than 1.0.

13. The composition of any of claims 1-12, wherein the molar ratio of aptamer to molecular payload is at least 2.0.

14. The composition of any of claims 1-13, wherein the molar ratio of aptamer to molecular payload is at least 2.0, at least 3.0, at least 4.0, at least 5.0, at least 10.0, at least 15.0, at least 20.0, at least 25.0, at least 30.0, at least 35.0, or at least 40.0.

15. The composition of any of claims 1-14, wherein the molar ratio of aptamer to molecular payload is from 1.0-20.0, 1.0-40.0, 1.1-20.0, 1.1-40.0, 2.0-20.0, or 2.0-40.0.

16. The composition of any of claims 1-15, wherein the molecular payload is not covalently bound to the aptamer.

17. The composition of any of claims 1-16, wherein the aptamer is not a targeting moiety.

18. The composition of any of claims 1-17, wherein the aptamer is not a therapeutic agent.

19. The composition of any of claims 1-18, wherein the molecular payload is hydrophilic (e.g., Log D or Log P<0).

20. The composition of any of claims 1-18, wherein the molecular payload is a therapeutic agent.

21. The composition of any of claims 1-20, wherein the molecular payload is a small molecule or a biologic.

22. The composition of any of claims 1-21, wherein the molecular payload is a small molecule.

23. The composition of any of claims 1-22, wherein the molecular payload is an anesthetic, an antibiotic, or a neurotransmitter.

24. The composition of any of claims 1-23, wherein the molecular payload is an anesthetic.

25. The composition of any of claims 1-24, wherein the molecular payload is a site 1 sodium channel blocker.

26. The composition of any of claims 1-25, wherein the molecular payload is tetrodotoxin or saxitoxin.

27. The composition of any of claims 1-23, wherein the molecular payload is an antibiotic.

28. The composition of any of claims 1-23, wherein the molecular payload is kanamycin.

29. The composition of any of claims 1-23, wherein the molecular payload is a neurotransmitter.

30. The composition of any of claims 1-23, wherein the molecular payload is serotonin.

31. The composition of any of claims 1-21, wherein the molecular payload is a polypeptide.

32. The composition of any of claims 1-21, wherein the molecular payload is insulin.

33. The composition of any of claims 1-32, comprising an effective amount of the molecular payload.

34. The composition of any of claims 1-33, comprising a therapeutically effective amount of the molecular payload.

35. The composition of any of claims 1-34, further comprising a pharmaceutically acceptable excipient.

36. The composition of any of claims 1-35, further comprising a therapeutic agent (e.g., epinephrine).

37. The composition of any of claims 1-36, wherein the composition is characterized in that, when tested in vitro by placing the composition in a medium, less than 100%, less than 90%, less than 80%, less than 70%, less than 60%, less than 50%, less than 40%, less than 30%, less than 20%, or less than 10% of the molecular payload is released from the composition 24 hours after placing the composition in the medium.

38. The composition of any of claims 1-37, wherein the composition is characterized in that, when administered to a subject, less than 100%, less than 90%, less than 80%, less than 70%, less than 60%, less than 50%, less than 40%, less than 30%, less than 20%, or less than 10% of the molecular payload is released from the composition 24 hours after administration of the composition.

39. The composition of any of claims 1-38, wherein the composition is characterized in that, when administered to a subject, duration of a therapeutic effect is extended by at least 10%, at least 20%, at least 30%, at least 40%, at least 50%, at least 60%, at least 70%, at least 80%, at least 90%, at least 100%, or at least 200% compared to duration of the therapeutic effect upon administration of the molecular payload alone or in a composition without the aptamer.

40. The composition of claim 39, wherein the therapeutic effect is nerve block and/or reduced pain.

41. The composition of any of claims 1-40, wherein the composition is a depot for the molecular payload.

42. A method of treating a disease or condition, the method comprising administering an effective amount of the composition of any of claims 1-41 to a subject in need thereof.

43. The method of claim 42, wherein less than 100%, less than 90%, less than 80%, less than 70%, less than 60%, less than 50%, less than 40%, less than 30%, less than 20%, or less than 10% of the molecular payload is released from the composition 24 hours after administration of the composition to the subject.

44. The method of claim 42 or 43, wherein duration of a therapeutic effect is extended by at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1,000%, or at least 10,000% compared to duration of a therapeutic effect upon administration of the molecular payload alone or in a composition without the aptamer.

45. The method of claim 44, wherein the therapeutic effect is nerve block and/or reduced pain.

46. The method of any of claims 42-45, wherein the disease or condition is pain.

47. The method of any of claims 42-44, wherein the disease or condition is an infection, tuberculosis, or diabetes.

48. The method of any of claims 42-47, wherein the subject is a mammal.

49. The method of any of claims 42-48, wherein the subject is a human.

50. A method of preparing the composition of any of claims 1-41, the method comprising providing a molecular payload; identifying or preparing an aptamer that binds to the molecular payload; and combining the molecular payload and aptamer in a composition.

51. A kit comprising the composition of any of claims 1-41 and instructions for use.


SEQUENCE LISTING (11 sequences)

SEQ ID NO: 1 - DNA, 30 bp, synthetic construct
  source 1..30: mol_type = other DNA
aaaaatttcacacgggtgcctcggctgtcc

SEQ ID NO: 2 - DNA, 30 bp, synthetic construct; identical to SEQ ID NO: 1
  source 1..30: mol_type = other DNA
  modified_base 1..30: mod_base = OTHER; note = phosphorothioate linkage

SEQ ID NO: 3 - DNA, 30 bp, synthetic construct
  source 1..30: mol_type = other DNA
  modified_base 1..30: mod_base = OTHER; note = phosphorothioate linkage
ggctccactcatacttccgagtaagacggt

SEQ ID NO: 4 - DNA, 30 bp, synthetic construct
  source 1..30: mol_type = other DNA
  modified_base 1..30: mod_base = OTHER; note = phosphorothioate linkage
ggacagccgaggcacccgtgtgaaattttt

SEQ ID NO: 5 - DNA, 34 bp, synthetic construct
  source 1..34: mol_type = other DNA
  modified_base 1..34: mod_base = OTHER; note = phosphorothioate linkage
ttgagggtcgcatcccgtggaaacaggttcattg

SEQ ID NO: 6 - DNA, 44 bp, synthetic construct
  source 1..44: mol_type = other DNA
cgactggtaggcagataggggaagctgattcgatgcgtgggtcg

SEQ ID NO: 7 - DNA, 44 bp, synthetic construct; identical to SEQ ID NO: 6
  source 1..44: mol_type = other DNA
  modified_base 1..44: mod_base = OTHER; note = phosphorothioate linkage

SEQ ID NO: 8 - DNA, 21 bp, synthetic construct
  source 1..21: mol_type = other DNA
tgggggttgaggctaagccga

SEQ ID NO: 9 - DNA, 21 bp, synthetic construct
  source 1..21: mol_type = other DNA
  modified_base 1..21: mod_base = OTHER; note = phosphorothioate linkage
tgggggttgaggctaagccga

SEQ ID NO: 10 - DNA, 30 bp, synthetic construct
  source 1..30: mol_type = other DNA
ggtggtggggggggttggtagggtgtcttc

SEQ ID NO: 11 - DNA, 30 bp, synthetic construct; identical to SEQ ID NO: 10
  source 1..30: mol_type = other DNA
  modified_base 1..30: mod_base = OTHER; note = phosphorothioate linkage